(19) United States
(12) Patent Application Publication
(10) Pub. No.: US 2019/0025314 A1
(43) Pub. Date: Jan. 24, 2019

(54) METHODS FOR DETERMINING TUMOUR PHENOTYPES

(71) Applicants: Janette Dixon, Goldenhill Park (SG); John W. Holaday, Bethesda, MD (US)

(72) Inventors: Janette Dixon, Goldenhill Park (SG); John W. Holaday, Bethesda, MD (US)

(21) Appl. No.: 16/068,889

(22) PCT Filed: Jan. 11, 2017
(86) PCT No.: PCT/IB2017/050128

§ 371 Date: Jul. 9, 2018

Related U.S. Application Data
(60) Provisional application No. 62/277,446, filed on Jan. 11, 2016.

(51) Int. Cl. G01N 33/574; C12Q 1/6886

(52) U.S. Cl. CPC G01N 33/57488; C12Q 1/6886; G01N 2333/70539; C12Q 2600/106; C12Q 2600/156; C12Q 2600/154; C12Q 2600/118; C12Q 2600/158

(57) ABSTRACT

The present invention relates to a method of determining the major histocompatibility complex profile of a tumour subject with cancer comprising the steps of: a) obtaining a physiological fluid sample comprising texosomes or a texosome-enriched sample of extracellular vesicles from a physiological fluid sample of the subject; and b) identifying the phenotype of texosomes associated with tumour cells having aberrant MHC-I expression to determine the major histocompatibility complex profile of the tumour of the subject; as well as various stratifications for immunotherapy, methods of prognosis of cancer in a subject and methods of treatment based on the MHC-I or HLA-G expression profile.

### FIELD OF INVENTION

The invention relates to methods of determining the phenotypes of the total tumour burden of a subject undergoing cancer immunotherapy. In particular, although not exclusively, the invention relates to methods of determining and monitoring the major histocompatibility complex (MHC) phenotypes of the total tumour burden of the subject.

### BACKGROUND ART

Cancer immunotherapy is a new paradigm in the treatment of subjects with cancer. In the 1980s a new protein receptor on the surface of T-cells was identified; cytotoxic T-lymphocyte antigen 4 (CTLA-4). The function of CTLA-4 was to prevent T-cells launching full-out immune attacks. It was postulated that blocking CTLA-4 might set the immune system free to destroy cancer. It was subsequently demonstrated that antibodies to CTLA-4 could erase tumours in mice. Since those studies in the 1990s, antibodies to CTLA-4 and other T-cell expressed molecules such as PD-1 have now reached the clinic. In addition to the use of these “checkpoint inhibitors” (as they have become known), other aspects of cancer immunotherapy include the use of cancer vaccines such as dendritic cell vaccines or peptide vaccines, which are designed to stimulate anti-tumour cytotoxic T lymphocytes following administration, and adoptive T cells therapies such as chimeric antigen receptor T cell therapy (CAR-T) therapy or TCR Therapy.

In order to have an anti-tumour effect, cytotoxic T-cells require neoantigen presentation on the target cell by the major histocompatibility complex (MHC). The MHC multi-gene family encodes a series of cell surface proteins that function to bind and present antigens. MHC class I is expressed by all nucleated cells and together with the β2 microglobulin chain functions to display short peptide antigens derived from either intracellular pathogens or endogenous self-antigens. MHC class II is expressed by a specialized group of immune cells that present antigens derived from extracellular pathogens.

One of the means by which tumour cells evade the host immune response is by the down-regulation of MHC class I molecule expression. The anti-tumour response of any endogenous or administered T-cells is thereby circumvented. Several mechanisms of down-regulation of MHC class I molecule expression have been characterised and classified as one of seven phenotypes (Garcia-Lora et al (2003)). These phenotypes may be further classified as “hard” or “soft” according to whether the underlying escape mechanism has the potential to be reversed. For example, down-regulation attributable to the hypermethylation of DNA may be reversed by the administration of agents such as azacitidine and decitabine.

Being able to determine the MHC class I phenotype of the tumour burden of a subject is therefore of importance in both identifying subjects likely to be responsive to a proposed treatment regimen, and monitoring for the development of phenotypes that may require modification of the existing treatment regimen or be indicative of tumour progression. The MHC class I phenotype of the tumour cell of a tissue sample may be surveyed using a panel of anti-MHC antibodies. In addition to immunohistology, microdissection may be employed to obtain samples of DNA and RNA (Garrido et al (2010)).

A limitation of the foregoing methods is that the sampling is both spatially and temporally discrete. Numerous samples need to be obtained and analysed to approach what may be regarded as a determination of the MHC expression profile of the total tumour burden of the subject.

A primary tumour may contain clones with diverse MHC phenotypes, but an understanding of this heterogeneity is incomplete without analysis of MHC diversity in different metastases. MHC-I molecules in different metastatic colonies may frequently acquire new alterations during malignant dissemination, generating novel MHC-I phenotypes and providing the basis for additional cancer heterogeneity. However, it is a challenging task to trace each metastatic node and obtain multiple biopsies and tumour cell lines from distant lesions in humans for the study of MHC expression patterns in metastatic dissemination. In addition, not all primary tumours are accessible for biopsy, or multiple biopsies as would be required to determine the extent of heterogeneity of MHC-I expression.

It is an object of the present invention to provide a method that facilitates the determination of the MHC expression profile of the total tumour burden of a subject. It is an object of the present invention to provide a method that more accurately determines the MHC expression profile of the total tumour burden of a subject. These objects are to be read in the alternative with the object at least to provide a useful choice in the selection of such methods.

The term “texosome” refers to an extracellular vesicle (such as an exosome, microvesicle, oncosome). Suitably, texosomes may comprise tumour polypeptides, tumour DNA and/or tumour RNA, and includes extracellular vesicles derived from particular types of cancers, including, but not limited to prostasomes. An exosome is created intracellularly when a segment of the cell membrane spontaneously invaginates and is endocytosed. The internalized segment is broken into smaller vesicles that are subsequently expelled from the cell. The latter stage occurs when the late endosome, containing many small vesicles, fuses with the cell membrane, triggering the release of the vesicles from the cell. The vesicles (once released are called “exosomes”) consist of a lipid raft embedded with ligands, i.e., polypeptides, common to the original cell membrane. Exosomes secreted by cells under normal and pathological conditions contain proteins, DNA and functional RNA molecules, including mRNA and miRNA, which can be shuttled from one cell to another, affecting the recipient cell's protein production. Microvesicles are formed by blebbing outwards from a cell's surface, and as they are formed the microvesicles carry with them membrane embedded with polypeptides from the parent cell.

### STATEMENT OF INVENTION

Advantageously, the present invention provides methods for determining the major histocompatibility complex profile of the total tumour burden of a subject with cancer by the characterisation of texosomes. Without wishing to be bound by theory, the present invention is, in part, predicated on the surprising finding that MHC profiling of texosomes correlates with MHC profiling of tumour cells from with the texosomes are derived. Therefore, a physiological fluid of a subject can be used to determine a representative MHC profile of the total tumour burden of the subject. Accordingly, the present invention may advantageously provide improved methods for determining the major histocompatibility complex profile of the total tumour burden of a subject with cancer.

In one aspect, the present invention relates to a method of determining the major histocompatibility complex profile of a tumour of a subject with cancer comprising the steps of:

a) obtaining i) a physiological fluid sample comprising texosomes or ii) a texosome-enriched sample of extracellular vesicles from a physiological fluid sample, of the subject;

b) identifying the phenotype of texosomes associated with tumour cells having aberrant

MHC-I expression to determine the major histocompatibility complex profile of the total tumour burden of the subject.

Suitably, the method may determine the major histocompatibility profile of the total tumour burden of the subject.

Suitably, the method may further comprise identifying the phenotype of texosomes associated with tumour cells that have normal MHC-I expression.

Suitably, the step of determining the MHC-I phenotype of texosomes associated with tumour cells may determine the level of:

a) texosomes associated with tumour cells having normal MHC-I expression

b) texosomes associated with tumour cells having aberrant MHC-I expression which is reversible with cytokines; and/or

c) texosomes associated with tumour cells having aberrant MHC-I expression which is irreversible with cytokines.

Suitably, the subject may be a human subject. When the subject is a human subject the terms “MHC-I” and “HLA-I” as used are interchangeable.

Suitably, the step of identifying the phenotype of texosomes associated with tumours cells having aberrant MHC-I (HLA-I) expression may determine the level of texosomes associated with tumour cells having one or more (or any combination thereof) of the following phenotypes:

Phenotype I—total loss of HLA class I expression;

Phenotype II—loss of HLA class I haplotype;

Phenotype III—loss of an HLA class I locus;

Phenotype IV—HLA class I allelic loss;

Phenotype V—a compound pheno

type;

Phenotype VI—unresponsive to interferons; and

Phenotype VII—downregulation of classical HLA molecules with aberrant expression of non-classical HLA molecules.

Suitably, the physiological fluid sample may be: a blood sample, ascites, pleural effusion, cerebrospinal fluid or urine of the subject.

Suitably, the determining step may comprise the use of exome sequencing; flow cytometry; mass cytometry or any combination thereof.

Suitably, the method may comprise comparing the MHC-I profile of the subject's peripheral blood cells to the MHC-I profile of the texosomes with an aberrant MHC-I profile. Suitably, the comparison may identify one or more (or two or more) of the following:

a) Differences in locus expression;

b) Differences in allelic expression;

c) B2m gene alteration; and

d) Loss of heterozygosity in chromosome 6 and/or chromosome 15.

Suitably, the determining step may comprise using a panel of antibodies directed against different loci and allelic specificities.

Suitably, the expression of one or more of the following on the texosomes is also measured: TAP 1, TAP2, tapasin, calnexin, calreticulin, LMP2 and LMP7.

Suitably, the expression of HLA-G may also be measured.

Suitably, the texosomes may be exosomes.

Suitably, the subject with cancer may have a solid tumour or the subject may have a haematological tumour. Suitably, the subject may have a cancer selected from the group consisting of: leukemia, brain cancer, prostate cancer, liver cancer, ovarian cancer, stomach cancer, colorectal cancer, throat cancer, breast cancer, skin cancer, melanoma, lung cancer, sarcoma, cervical cancer, testicular cancer, bladder cancer, endocrine cancer, endometrial cancer, esophageal cancer, glioma, lymphoma, neuroblastoma, osteosarcoma, pancreatic cancer, pituitary cancer, renal cancer, nasopharyngeal cancer.

In another aspect, the present invention relates to a method for monitoring cancer progression in a subject, said method comprising:

a) determining the major histocompatibility complex profile of the total tumour burden of said subject with cancer in accordance with the method of determining the major histocompatibility complex profile of the invention; and

b) Repeating step a) and comparing the major histocompatibility complex profile of the total tumour burden of said subject with the major histocompatibility complex profile of the total tumour burden of said subject determined in step a).

In a further aspect, the present invention relates to a method for determining the prognosis of a subject with cancer, said method comprising:

a) determining the major histocompatibility complex profile of the total tumour burden of said subject with cancer in accordance with the method of determining the major histocompatibility complex profile of the invention; and

b) repeating step a) and comparing the major histocompatibility complex profile of the total tumour burden of said subject with the major histocompatibility complex expression profile of the total tumour burden of said subject determined in step a;

wherein an increase in texosomes associated with tumour cells having aberrant MHC-I expression which is irreversible with cytokines is indicative of poor prognosis.

In another aspect, the present invention relates to a method for determining the therapeutic effect of a medicament used in the treatment of cancer, said method comprising:

a) determining the major histocompatibility complex profile of the total tumour burden of said subject with cancer from a physiological sample of the subject prior to treatment with a medicament in accordance with the method of determining the major histocompatibility complex profile of the invention;

b) determining the major histocompatibility complex profile of the total tumour burden of said subject with cancer, in accordance with the method of step a), from a physiological sample of the subject obtained after treatment with a medicament for a predetermined period; and

c) comparing the major histocompatibility complex profiles obtained in steps a) and b).

In a further aspect, the present invention relates to the use of an isolated sample of texosomes of a subject with cancer to predict responsiveness or sensitivity of said subject to treatment with cytokine therapy or HDAC inhibitors.

Suitably, the MHC class I expression profile of texosomes isolated from a physiological fluid of the subject may be measured.

In another aspect, the present invention relates to a method of increasing the sensitivity rate of an immunotherapy to treat cancer in a patient population, said method comprising selecting a subpopulation, wherein the major histocompatibility complex expression profile of the total tumour burden has been determined using texosome analysis in the subpopulation and said subpopulation has:

a) a level below a predetermined threshold of texosomes associated with tumour cells unresponsive to cytokines.

In yet another aspect, the present invention relates to a method of identifying a subject having increased likelihood of responsiveness or sensitivity to an immunotherapy, said method comprising:

a) determining the level of texosomes associated with tumour cells that are unresponsive to cytokines;

Wherein a level below a predetermined threshold is indicative of having an increased likelihood of responsiveness or sensitivity to cytokine therapy.

In a further aspect, the present invention relates to a method of identifying a subject having responsiveness or sensitivity to an immunotherapy, said method comprising:

a) determining the level of texosomes associated with tumour cells that are unresponsive to cytokines;

Wherein a level below a predetermined threshold is indicative of having responsiveness or sensitivity to the immunotherapy.

Suitably, the immunotherapy may be any immunotherapy that leads to increased cytokine production, including the following: cytokine therapy, dendritic cell vaccines, adoptive T cell vaccines, peptide vaccines, checkpoint inhibitors and antibodies which induce the release of cytokines.

In another aspect, the present invention relates to immunotherapy (e.g. cytokine therapy) or HDAC inhibitors for use in the treatment of cancer is a subject, wherein the subject has undergone texosome MHC-I profile analysis.

In a further aspect, the present invention relates to a method of increasing the sensitivity rate of a cancer vaccine utilising a HLA-A2 specific peptide to treat or prevent cancer in a patient population, said method comprising selecting a subpopulation, wherein the major histocompatibility complex profile of the total tumour burden has been determined using texosome analysis in the subpopulation and said subpopulation has:

a) a level below a predetermined threshold of texosomes associated with tumour cells having a loss of HLA-A2 expression.

In a further aspect, the present invention relates to a method of identifying a subject having increased likelihood of responsiveness or sensitivity to a cancer vaccine utilising a HLA-A2 specific peptide, said method comprising:

a) determining the level of texosomes associated with tumour cells having a loss of HLA-A2 expression;

Wherein a level below a predetermined threshold is indicative of having an increased likelihood of responsiveness or sensitivity to a cancer vaccine utilising a HLA-A2 specific peptide.

In yet another aspect, the present invention relates to a method of identifying a subject having responsiveness or sensitivity to a cancer vaccine utilising a HLA-A2 specific peptide, said method comprising:

a) determining the level of texosomes associated with tumour cells having a loss of HLA-A2 expression;

Wherein a level below a predetermined threshold is indicative of having responsiveness or sensitivity to a cancer vaccine utilising a HLA-A2 specific peptide.

In another aspect, the present invention relates to cytokine therapy, a cancer vaccine or a HDAC inhibitor for use in the treatment or prevention of cancer is a subject, wherein the subject has undergone texosome MHC-I expression profile analysis.

In a further aspect, the present invention relates to a method of determining a treatment regime for a subject having cancer, said method comprising:

a) determining the major histocompatibility complex profile of the total tumour burden of said subject with cancer in accordance with the method of determining the major histocompatibility complex profile of the invention;

b) administering a medicament based on the major histocompatibility complex profile of the total tumour burden of said subject.

In a yet further aspect, the present invention relates to a method of treating cancer in a subject identified as having an increased likelihood of responsiveness or sensitivity to immunotherapy by a method of the invention, comprising:

a) administering a therapeutically effective amount of the immunotherapy.

In another aspect, the present invention relates to a method of treating or preventing cancer in a subject identified as having an increased likelihood of responsiveness or sensitivity to cancer vaccines utilising HLA-A2 specific peptides by a method of the invention, comprising:

a) administering a therapeutically effective amount of a cancer vaccine utilising HLA-A2 specific peptide.

In a further aspect, the present invention relates to an isolated and enriched population of texosomes for which the MHC-I profile has been determined in accordance with a method of the invention for use in diagnostics.

In another aspect, the present invention relates to the use of an isolated and enriched population of texosomes for which the MHC-I profile has been determined in accordance with a method of the invention for:

a) diagnosis;

b) prognosis;

c) determining treatment; or

d) preparing modulated texosomes.

In a further aspect, the present invention relates to a method of determining the HLA-G expression profile of a tumour of a subject with cancer comprising the steps of:

a) obtaining a physiological fluid sample comprising texosomes or a texosome-enriched sample of extracellular vesicles from a physiological fluid sample of the subject;

b) identifying the phenotypes of texosomes associated with tumour cells having HLA-G expression.

### DETAILED DESCRIPTION

In the description and claims of this specification the following acronyms, phrases and terms (and their paronyms) have the meaning provided: “comprising” means “including”, “containing” or “characterized by” and does not exclude any additional element, ingredient or step; “consisting essentially of” means excluding any element, ingredient or step that is a material limitation; “consisting of” means excluding any element, ingredient or step not specified except for impurities and other incidentals; “EV” means extracellular vesicle; “exosome” means a sub-type of extracellular vesicle arising from the endosomal network and ranging in size from 30 to 100 nm; “expression profile” means the distribution in a population of a range of phenotypes; “extracellular vesicles” means cell-secreted vesicles ranging in size from 30 to 2,000 nm; “microvesicles” means extracellular vesicles arising from direct budding from the plasma membrane and ranging in size from 50 to 2,000 nm; “population” means belonging to the same type or sub-type; and “texosome” means an extracellular vesicle derived from a tumour cell.

The terms “first”, “second”, “third”, etc. used with reference to elements, features or integers of the subject matter defined in the Statement of Invention and Claims, or when used with reference to alternative embodiments of the invention are not intended to imply an order of preference. Where concentrations or ratios of reagents or solvents are specified, the concentration or ratio specified is the initial concentration or ratio of the reagents or solvents. Where values are expressed to one or more decimal places standard rounding applies. For example, 1.7 encompasses the range 1.650 recurring to 1.749 recurring.

One of the means by which tumour cells evade the host immune response is by the down-regulation of MHC class I molecule expression (Aptsiauri et al (2013)). The anti-tumour response of any endogenous or administered T-cells is thereby circumvented. This down-regulation has been reported in up to 90% of certain types of human cancers (Garrido 1997), and is widely used by tumour cells to evade T cell-mediated recognition and destruction. MHC class I expression plays an important role in the interaction between the expression of tumour supressing genes and cancer immunogenicity (Garrido 2016). MHC class I molecules are heterodimers that consist of two polypeptide chains, α and β2-microglobulin (β2m). The two chains are linked noncovalently via interaction of β2m and the α3 domain. Only the a chain is polymorphic and encoded by a HLA gene, while the β2m subunit is not polymorphic and encoded by the Beta-2 microglobulin gene. The α3 domain is plasma membrane-spanning and interacts with the CD8 co-receptor of T-cells. The α3-CD8 interaction holds the MHC I molecule in place while the T cell receptor (TCR) on the surface of the cytotoxic T cell binds its α1-α2 heterodimer ligand, and checks the coupled peptide for antigenicity. The al and α2 domains fold to make up a groove for peptides to bind. MHC class I molecules bind peptides that are 8-10 amino acid in length (Garcia-Lora 2003).

The human leukocyte antigen (HLA) system or complex is a gene complex encoding the MHC proteins in humans. HLA genes are highly polymorphic, which means that they have many different alleles, allowing them to fine-tune the adaptive immune system. The proteins encoded by certain genes are also known as antigens, as a result of their historic discovery as factors in organ transplants. Different classes have different functions (Garcia-Lora 2003):

HLAs corresponding to MHC class I (A, B, and C) present peptides from inside the cell. For example, if the cell is infected by a virus, the HLA system brings fragments of the virus to the surface of the cell so that the cell can be destroyed by the immune system. These peptides are produced from digested proteins that are broken down in the proteasomes. MHC class I proteins associate with β2m, which unlike the HLA proteins is encoded by a gene on chromosome 15.

HLAs corresponding to MHC class II (DP, DM, DOA, DOB, DQ, and DR) present antigens from outside of the cell to T-lymphocytes. These particular antigens stimulate the multiplication of T-helper cells, which in turn stimulate antibody-producing B-cells to produce antibodies to that specific antigen. Self-antigens are suppressed by regulatory T cells.

HLAs corresponding to MHC class III encode components of the complement system.

MHC Class I-deficient tumour variants may lose the antigen-presenting molecule and are, therefore, resistant to T cell cytotoxicity. At the same time, cells with total loss of MHC Class I surface expression can potentially become susceptible to natural killer (NK) cell antitumour activity induced by the lack of inhibitory MHC antigens (“missing self”). Another immunoselection route is provided by the partial loss of HLA Class I antigens that allows tumour cells to escape both CTL and NK attack. For instance, it has been reported that colorectal cancer with inter-mediate HLA Class I expression are associated with poor prognosis as compared to HLA positive or totally negative tumours, suggesting that such tumours with intermediate HLA Class I expression may avoid both NK- and T cell-mediated immune surveillance.

The secretion of extracellular vesicles facilitates the remodelling of cellular membranes, recycling and the removal of cellular components. The secretion of extracellular vesicles also mediates intercellular communication and transport of membrane bound receptors, nucleic acids and other proteins, including major histocompatibility complex molecules. The secretion of extracellular vesicles from tumour cells facilitates modification of the extracellular matrix, the vesicles being enriched for matrix degrading metalloproteases, and play a critical role in modulating the migration and invasion of tumour cells. Extracellular vesicles secreted by tumour cells (“texosomes”) are not anatomically compartmentalised and can therefore be isolated by relatively non-invasive methods from the blood, cerebrospinal fluid and other physiological fluids of subjects with cancer.

The present invention correlates the MHC profile of texosomes to the MHC profile of tumour cells from which the texosomes are derived. Accordingly, the major histocompatibility complex (MHC) phenotypes (class I and II) of the entire tumour burden of the subject, i.e. the MHC expression profile, may therefore be determined and conveniently monitored over time by determining the phenotype of texosomes associated with tumour cells having aberrant MHC-I expression.

Methods of determining the major histocompatibility complex profile of the total tumour burden of a subject with cancer

Methods disclosed herein are directed to determining the major histocompatibility complex (hereinafter MHC) profile of at least one tumour of a subject with cancer comprising the step of obtaining a sample or samples from a subject with cancer, the sample or samples comprising extracellular vesicles released by tumour cells (“texosomes”) and determining the MHC expression profile of the texosomes In certain embodiments, the methods disclosed herein may provide a MHC profile of the total tumour burden present in a subject, including primary tumours and any metastases. The samples may be obtained periodically over a defined time course. In certain example embodiments, the cancer is lung cancer. In certain example embodiments, the sample is a pleural effusion sample. In one example embodiment, the major histocompatibility complex (“MHC”) expression profile comprises a major MHC I and MHC II profile. In another example embodiment, the MHC expression profile comprises a MHC I expression profile. In certain example embodiments, the MHC expression profile consists only of a MHC I expression profile.

In one aspect, the present invention relates to a method of determining the major histocompatibility complex profile of a tumour of a subject with cancer comprising the steps of:

a) obtaining i) a physiological fluid sample comprising texosomes or ii) a texosome-enriched sample of extracellular vesicles from a physiological fluid sample, of the subject;

b) identifying the phenotype of texosomes associated with tumour cells having aberrant MHC-I expression to determine the major histocompatibility complex profile of the total tumour burden of the subject.

Advantageously, this provides a straightforward way to determine the MHC prolife of a subject.

By utilising a physiological fluid the need for biopsies may also advantageously be avoided and the regular monitoring of cancer progression can be achieved.

As used herein “tumour cells having normal MHC-I expression” refers to tumour cells having MHC-I which substantially corresponds to MHC-I expression of a normal or otherwise healthy cell of a peripheral blood cell or a cell of the same type as the tumour cell. For example, where the tumour cell is found in the lung a normal MHC-I expression profile may be one that substantially corresponds to that of a healthy lung cell. Thus, the MHC-I profile of the tumour cell may be compared to a peripheral blood cell and/or a healthy cell of the same type to determine if the tumour cell has a normal MHC-I profile. By “substantially corresponds” in this context it is meant that the MHC-I expression of the tumour cells is within the normal or physiologically normal variability seen between healthy cells of the same type.

As used herein “tumour cells having an aberrant MHC-I expression” refers to tumour cells which do not have a normal MHC-I expression compared to a normal or otherwise healthy or physiologically normal cell of a peripheral blood cell or a cell of the same type as the tumour cell. Typically, aberrant tumour cells have a down regulation of MHC-I expression. Suitably, the down regulation may be at least 5% or at least 10% or at least 15% or at least 20% or at least 25% of the expression of a normal or otherwise healthy cell of a peripheral blood cell or a cell of the same type as the tumour cell.

Suitably, the method may further comprise identifying the phenotype of texosomes associated with tumour cells that have normal MHC-I expression.

Suitably, the texosomes may be isolated from the physiological fluid sample prior to determining the MHC expression profile. For example, a fraction of the sample containing the texosomes may be obtained using ultracentrifugation, size exclusion chromatography or precipitation, for example. In addition or in the alternative, the texosomes may be further isolated by affinity means. For example, the texosomes may be extracted from the sample, with or without ultracentrifugation, using one or more tumour-specific antigens. For example, the texosomes may be isolated by incubation with paramagnetic beads comprising one or more tumour-specific antigens.

The paramagnetic bead to texosome ratio may be any ratio equal to or greater than 1. For example, the ratio of paramagnetic beads to texosomes may be 3:1 or 2:1.

Suitably, the beads may be added at a limiting bead to texosome ratio. Suitably the beads may be added at a 1:1 paramagnetic bead to texosome ratio.

Whilst paramagnetic beads are specifically recited herein, the skilled person would be readily aware of other similar bead-based isolation techniques and such techniques can be used in accordance with the present invention.

Suitably, the texosomes may be isolated by affinity chromatography using antibodies using e.g. tumour specific antigens. In addition or in the alternative, the texosomes may be isolated using ion exchange chromatography as texosomes have different charges to EVs from healthy cells. Other known affinity isolation methods suitable for the isolation of texosomes are also contemplated.

Suitably, the texosome-enrichment process increases the texosomes per 100 EVs by at least two-fold, or at least three-fold or at least 5-fold or at least 10-fold. Suitably, the percentage of EVs that are texosomes in the texosome-enriched sample is at least 15% or at least 20% or at least 30% or at least 40% or at least 50%.

Suitably, the texosome-enriched sample of extracellular vesicles may be free or substantially free of extracellular vesicles (e.g. exosomes) which have not been released by tumour cells. For example, there may be a sample in which at least 90% or at least 95% or at least 96% or at least 97% or at least 98% or at least 99% of the extracellular vesicles have been released by tumour cells.

A “texosome associated with tumour cells having aberrant MHC-I expression” may be an extracellular vesicle (e.g. an exosome) released by a tumour cell which has aberrant MHC expression. For example, the tumour cell may have partial or total loss of MHC-I expression.

Presently, a precise definition of the tumour cell having aberrant MHC expression and the underlying mechanism of HLA Class I defects can be obtained by the combined use of immunohistochemistry with tissue microdissection, polymerase chain reaction (PCR), comparative genomic hybridization, fluorescence in situ hybridization and loss of heterozygosity (LOH) analysis with specific markers spanning the chromosomal region of interest (Garrido 2010).

Different mechanisms can lead to the total or partial loss of HLA expression. The molecular mechanisms that have been found to underlie these alterations in MHC Class I expression vary and can occur at any step required for MHC synthesis, assembly, transport or expression on cell surface. These defects can occur at the genetic, epigenetic, transcriptional and post-transcriptional levels. They can be classified in a number of different (potentially overlapping) ways (Garrido 2010).

Suitably, determining the phenotype of texosomes associated with tumour cells having aberrant MHC-I expression, to determine the major histocompatibility complex profile of the total tumour burden of the subject, may involve determining the presence or absence of texosomes associated with tumour cells of a specific phenotype associated with down-regulation of MHC class I molecule expression or determining the level of such texosomes. Where the level is determined, this could be as a percentage of the total number of texosomes in the sample of a specific concentration.

Suitably, the presence or absence of or the level of texosomes associated with tumour cells of two or more specific phenotypes associated with down-regulation of MHC class I molecule expression may be determined.

Tumour derived extracellular vesicles include tumour derived exosomes, tumour derived microvesicles, tumour derived oncosomes and tumour derived apoptotic bodies. Exosomes are formed by budding inwards of the cell membrane to form a vesicle within a multivesicular body, which later fuses with the cell membrane to pour its contents (exosomes) outside of the cell. Microvesicles are formed directly by blebbing off the surface of the cell. In both cases the membrane of the exosomes and microvesicles are formed from the membrane of the surface of the cell they derive from. Thus, exosomes and microvesicles derived from tumour cells have a membrane formed from the membrane of the tumour cell.

Texosomes (e.g. exosomes) secreted by tumour cells are small MHC-bearing vesicles of endocytic origin which are released into the extracellular milieu (Miller and Grunewald, 2015). It is postulated that this MHC profile comes from the tumour cells from which they are derived from, because exosomes do not contain the machinery to manufacture proteins. Without wishing to be bound by theory, it is believed that the MHC molecules that come along with the membrane when a texosome is formed are representative of the tumour cells they come from i.e., that the same altered phenotypes are present on the texosomes and at the same ratios as they were found on the parent tumour cells.

Texosomes (e.g. exosomes) can be harvested from a physiological fluid (such as blood) and such a sample can be representative of the entire tumour burden of a subject.

Texosomes (e.g. exosomes) carry on their surface MHC-I molecules, and may comprise DNA and RNA from the tumour cells. Accordingly, the present invention has surprisingly found that texosomes can be used for phenotyping of the tumour cells from which they are released. Thereby, allowing from e.g. a blood sample, the MHC profile representative of the total tumour burden to be obtained.

For example, the MHC profile of a patient's peripheral blood cells may be compared to the HLA profile found on tumour exosomes to detect differences in locus or allelic expression or to detect β2m gen alteration or loss of heterozygosity (LOH) in chromosomes 6 and 15 etc. Further, the MHC cell surface expression profile of texosomes may be correlated to the MHC cell surface expression profile of the tumour cells from which the texosomes are released.

Quantitative RT-PCR may be used to measure the levels of HLA mRNA in tumour exosomes, whereas flow cytometry methods, both traditional and new improved versions adapted for microvesicle analysis, can be used for detection of HLA-I heavy chain variants and β2m proteins. A large panel of anti-HLA antibodies directed against different locus and allelic specificities is available. Therefore, Western blotting, ELISA or immunoprecipitation can also be employed to confirm the presence and the amounts of HLA-I molecules in tumour exosome specimens.

The same techniques can be used to analyze levels of other components of the antigen presentation machinery, e.g. TAP 1/2, tapasin, calnexin, calreticulin, LMP2/7. These methods and/or other methods known the skilled person can be employed to characterize HLA-I phenotypes in different patients to identify carriers of “hard” lesions and “soft” lesions. Thus, using tumour exosomes from blood and other body fluids represents an attractive approach for tumour HLA-I phenotyping.

Suitably, texosomes may display the same MHC class I profile of tumour cells from which they derive.

Suitably, the step of determining the phenotype of texosomes associated with tumours cell having aberrant MHC-I expression may determine the level of:

a) texosomes associated with tumour cells having aberrant MHC-I expression which is reversible with cytokines; and/or

b) texosomes associated with tumour cells having aberrant MHC-I expression which is irreversible with cytokines.

Suitably, the defects in MHC Class I expression may be classified into two main groups: reversible defects (regulatory or “soft”) and irreversible defects (structural or “hard”). Cells can be treated in vitro with various immunomodulators (i.e., interferons (IFNs)) or pharmacological agents in attempts to recover normal HLA expression, and how they respond to such treatments determines if the tumours are (i) capable of recovering or upregulating HLA Class I antigens after cytokine treatment or (reversible or “soft”); or (ii) those that cannot recover HLA I expression (structural or “hard”) (Garrido 2010). Both of these classifications have been used to characterise solid tumours, cancer cell lines and metastatic lesions, and classification strongly influences the likelihood of inducing tumour recognition by the recovery of MHC class I antigens (Garrido 2016).

Irreversible defects may result from defects in the HLA or β2m genes. The most frequent result from the LOH in chromosome 6 harbouring HLA-ABC genes. However, LOH in one chromosome 15 that carries the β2m gene, along with mutation in the other homologous gene, can also result in an irreversible MHC Class I defect. Although thought to be a rarer occurrence, irreversible genetic damage can also be caused by the selective loss of just one MHC Class I allele, due to mutations, deletions and somatic recombinations. Finally, mutations in genes related to the IFN-mediated signalling pathway are also classified as irreversible MHC Class I expression defects (Garrido 2010).

Suitably, irreversible defects may only be corrected by the replacement of the defective gene, such as with adenoviral-mediated recovery (Garrido 2010).

Reversible defects are those which are not structural, and can be corrected with cytokines or different agents. Downregulation of HLA Class I antigen expression is frequently caused by defects in the gene regulation of HLA Class I heavy chain genes, the β2m gene and the components of the antigen-processing machinery (APM). Such abnormal MHC Class I phenotypes show low mRNA levels of specific genes (heavy chain, b2m and APM) that seem to be coordinately downregulated. These defects are reversible, as they can be corrected in vitro by treatment with IFN-γ or other cytokines (Garrido 2010). HLA-A, HLA-B or HLA-C locus-specific downregulation is another frequent finding in tumour tissues and tumour cell lines of different origin. Low expression can be upregulated after IFN-γ treatment (Garrido 2010).

Epigenetic events associated with tumour development and with cancer progression have been found to underlie changes in HLA antigen and APM components. Unlike genetic alterations, epigenetic modifications can, in some cases, be reversed in vitro with pharmacologic agents that induce DNA hypomethylation or inhibit histone deacetylation (Garrido 2010).

Suitably, the subject may be a human subject. When the subject is a human subject the terms “MHC-I” and “HLA-I” as used are interchangeable.

Suitably, the step of determining the phenotype of texosomes associated with tumours cells having aberrant MHC-I (HLA-I) expression may determine the level of texosomes associated with tumour cells having one or more (or any combination thereof) of the following phenotypes:

Phenotype I—total loss of HLA class I expression;

Phenotype II—loss of HLA class I haplotype;

Phenotype III—Loss of an HLA class I locus;

Phenotype IV—HLA class I allelic loss;

Phenotype V—a compound phenotype;

Phenotype VI—unresponsive to interferons; and

Phenotype VII—Downregulation of classical HLA molecules with aberrant expression of non-classical HLA molecules.

Suitably, the method may be used to classify all texosomes associated with tumour cells having aberrant MHC-I expression into the seven phenotypes to determine a full expression profile for a subject. Alternatively, the method of the invention may be used to determine the level of texosomes associated with any one of the above-mentioned phenotypes, e.g. as a percentage of the total number of texosomes or as a percentage of the total number of texosomes associated with tumour cells having aberrant MHC profile. This may be advantageous where only texosomes of a specific phenotype are of interest. Suitably, the level of texosomes associated with tumour cells of any combination of the above-mentioned phenotypes may be measured individually (e.g. the level of texosomes associated with phenotype I and the level of texosomes associated with phenotype II may each be individually measured) or combined (e.g. the level of texosomes associated with phenotype I or phenotype II together may be determined).

Several mechanisms of down-regulation of MHC class I molecule expression have been characterised and classified as one of seven major phenotypes. These phenotypes are generally used in the art and the skilled person is readily aware of how to classify tumour cells into these seven phenotypes—see Garrido 2010, Garcia-Lora 2003 and Garrido 2001, for example. These citations describe:

Phenotype I refers to HLA Class I total loss of expression. This is a well-established altered HLA phenotype that can be produced by several molecular mechanisms (including β2m mutations, defects in the transporters associated with antigen processing (TAPs), or defects in the proteasome components of low-molecular-weight proteins (LMPs). Most of these cannot be regulated by cytokines.

Phenotype II refers to HLA haplotype loss; the loss of one of the two HLA haplotypes, due to loss of heterozygosity (LOH) in e.g. chromosome 6. This produces a HLA class I alteration that is present in most tumours, and can be difficult to determine due to contaminating stroma present in most tumour tissues analysed. Presently, it can precisely diagnosed using microdissected tumours and probably arises in early stages of tumour development. It can appear in combination with other HLA genetic lesions to produce compound phenotypes.

Phenotype III refers to HLA-A, -B or -C locus product downregulation. This altered phenotype is found when both products of HLA A, B, or C loci are co-ordinately downregulated. The mechanism of locus downregulation is often transcriptional.

Phenotype IV refers to loss of one single HLA class I allele. This alteration is defined as the loss of a single HLA class I allele.

Phenotype V refers to compound phenotypes i.e. of multiple other phenotypes. These result in a tumour cell expressing just one single HLA Class I allele. This requires a combination of at least two different alterations, for example a HLA haplotype loss and a HLA-B and -C locus downregulation (e.g. combining phenotypes II and III).

Phenotype VI refers to tumour cells unable to upregulate HLA Class I molecules due to a blockade in the interferon (IFN) signalling pathway. Some tumour cells express basal levels of HLA class I antigens, but have lost the capacity to upregulate these molecules in response to different cytokines, including α and γ interferons.

Phenotype VII refers to downregulation of classical HLA-A, -B and/or -C molecules combined with, at the same time, the appearance of HLA-E molecules. HLA-E is a nonclassical HLA class I molecule that produces a strong NK inhibition capacity after interaction with the CD94/NKGG2a immunity receptor.

The term “phenotype” as used herein is shorthand for phenotype and/or genotype.

In the context of the invention, as detailed above, the skilled person refers to different mechanisms which may downregulate MHC-I expression as different phenotypes irrespective of these being phenotypic or genotypic.

Accordingly, identifying the phenotype of texosomes associated with tumour cells having aberrant MHC-I expression may involve classifying a texosome into one of the seven “phenotypes” recited above.

The MHC phenotype of a metastatic colony not only shares the profile of the primary tumour, but can acquire new MHC alterations during dissemination, providing the basis for metastatic heterogeneity. Research into this is difficult due to difficulties in obtaining samples and processing samples into a sample possible to study, of both the primary tumour and any distant metastases from the same cancer patient. It is apparent that the accumulation of MHC class I alterations can not only play a role in cancer progression, but in metastatic dissemination.

There is also the possibility for intra-tumoural heterogeneity, where tumours can contain different cells with distinct variation in expression levels of different markers, or different MHC profiles. This may affect the ability of tumour cells to grow and metastasize (Garrido 2016).

Suitably, the physiological fluid may be any physiological fluid from the subject such as blood, urine, ascites, pleural effusion, cerebrospinal fluid (CSF). A person of ordinary skill in the art is readily able to select the appropriate physiological fluid comprising texosomes according to the type of cancer the subject has. For example, ascites may be used for ovarian cancer or liver cancer, metastasis or primary peritoneal carinomatosis. Suitably, ascites may be a particularly good source of texosomes for ovarian cancer. Likewise, pleural effusion is a good source of texosomes for e.g., lung cancer, CSF may be a good source of texosomes for e.g., gliobastoma, and urine may be a good source of texosomes for e.g., prostate cancer. A blood sample may be a good source of texosomes for determining the total tumour burden of a subject.

Methods of analysing tumour cells for MHC-I profiling are known in the art—see Garrido 2001, and Garrido 2010, for example. Such techniques can be used to profile texosomes in a similar manner.

Suitably, the identifying step may comprise the use of exome sequencing; flow cytometry; mass cytometry or any combination thereof. Suitably, the method may comprise comparing the MHC-I profile of the subject's peripheral blood cells or a heathy cell of the same type as the tumour cell to the MHC-I profile of the texosomes derived from a tumour cell. By routine methodology it is therefore possible to identify one or more (or two or more) of the following:

a) differences in locus expression;

b) differences in allelic expression;

c) B2m gene alteration; and

d) loss of heterozygosity in chromosome 6 and/or chromosome 15.

Suitably, the identifying step may comprise using a panel of known antibodies directed against different loci and allelic specificities. Various monoclonal antibodies directed to different HLA-I loci or allelic specificities may be employed. Some of which are referred to in Garrido 2001, for example.

Suitably, the expression of one or more of the following on the texosomes is also measured: TAP 1, TAP2, tapasin, calnexin, calreticulin, LMP2 and LMP7. These are all proteins associated with antigen presenting machinery. The expression of other such proteins may be determined in accordance with the method of the present invention.

Suitably, the texosomes may be exosomes.

Subject with cancer

As used here in the term “subject” refers to an individual, e.g., a human, pig, horse, mouse, cow, rat etc. having, or at risk of having, a proliferative disorder e.g. cancer. Suitably, the subject may have cancer.

Optionally, the subject is a subject having or at risk of having cancer, e.g. a solid cancer such as pancreatic cancer. The subject may be a patient i.e. a subject in need of treatment in accordance with the invention. The subject may have received treatment for the disorder. Alternatively, the subject may not have commenced treatment.

As used herein, the phrase “a proliferative disorder” is intended to include cancer, for example cancer of the lung, head and neck, brain, colon, rectum, oesophagus, stomach, liver, biliary tract, thyroid, kidney, cervix, ovary, uterus, skin, breast, bladder, prostate, pancreas and including haematological malignancies such as leukaemia, multiple myeloma and lymphoma. In particular, “a proliferative disorder” is intended to encompass solid tumours (cancers) (e.g. primary and recurrent solid tumours) such as pancreatic cancer, lung cancer, breast cancer, melanoma, colorectal cancer, ovarian cancer, gastric cancer, thyroid cancer, liver cancer and prostate cancer. A proliferative disorder of particular interest in the context of the invention is non-small-cell lung cancer.

Suitably, the cancer may be selected from the group consisting of: melanoma, prostate cancer, bladder cancer, lung cancer, non-Hodgkin's lymphoma and Hodgkin's lymphoma.

Suitably, the term “cancer” includes cancers of different tissues, and cancers at different stages including, but not limited to, leukemia, brain cancer, prostate cancer, liver cancer, ovarian cancer, stomach cancer, colorectal cancer, throat cancer, breast cancer, skin cancer, melanoma, lung cancer, sarcoma, cervical cancer, testicular cancer, bladder cancer, endocrine cancer, endometrial cancer, esophageal cancer, glioma, lymphoma, neuroblastoma, osteosarcoma, pancreatic cancer, pituitary cancer, renal cancer, nasopharyngeal cancer and the like.

As used herein, the term “tumour” refers to abnormal tissue masses, and includes both benign and malignant masses. Suitably, in some aspects the term “tumour” may refer to malignant masses.

Monitoring, prognosing and determining treatment efficacy

Advantageously, the profiling of texosomes in a physiological fluid of the subject can provide an easier and/or more accurate means of measuring cancer development, determining prognosis and measuring efficacy of treatment. It will be appreciated that regular analysis of a physiological fluid (such as urine or blood) is more clinically feasible than regularly obtaining and analysing biopsies.

Furthermore, as MHC-I profiling of texosomes comprised in a physiological fluid can reveal the MHC-I profile of the total tumour burden of a subject, such methods can be used to provide a more accurate method of monitoring cancer progress, making a prognosis and determining treatment efficacy.

In one aspect, the present invention provides a method for monitoring cancer progression in a subject, said method comprising:

a) determining the major histocompatibility complex profile of the total tumour burden of said subject with cancer in accordance with the method of invention and

b) Repeating step a) and comparing the major histocompatibility complex profile of the total tumour burden of said subject with the major histocompatibility complex profile of the total tumour burden of said subject determined in step a).

Suitably, an increase in texosomes associated with tumours cells having an aberrant MHC-I profile (such as those associated with hard lesions) may be indicative of the cancer progressing.

The “level of a texosomes associated with tumour cells having an aberrant MHC-I type” may refer to the amount of texosomes in a sample having a statistically significant downregulation MHC-I expression compared to a control. In this regard, the control may be a peripheral blood cell of the subject or a healthy cell of the same cell type. Suitably, the control in step a) is the same control for step b).

An increase in the number of texosomes on comparison of step b) with step a) refers to any statistically significant increase. Suitably, an increase of at least 5% or at least 10% or at least 20% may be indicative of poor prognosis. However, the skilled person is aware that this will depend on a number of factors, including the time period between the samples for step a) and step b). The shorter the time period the more significant an increase of a particular amount is.

Suitably, an increase in texosomes associated with tumours cells having aberrant MHC-I expression which is irreversible with cytokines may be indicative of the cancer progressing. Suitably, step b) is conducted at least 1 week, or at least 2 weeks, or at least 3 weeks, or at least a month, or at least 2 months or at least 3 months after step a).

Suitably, step b) may be repeated at subsequent time points to provide an ongoing analysis of cancer progression. For example, the step b) may be repeated on a monthly, 3-monthly or yearly basis.

Suitably, in each step a plurality of samples (i.e., from different physiological fluids) may be analysed and/or multiple samples of the same physiological fluid may be analysed to provide a more representative analysis of the MHC profile of the subject.

Suitably, steps a) and b) are repeated under similar (preferably identical conditions) to minimise variation from the analysis.

Suitably, a trend of decreasing texosomes with time (particularly where step b) is repeated one or more times) may be indicative of regression, whereas a trend of increasing texosomes may be indicative of cancer progression.

The accumulation of tumour cells carrying total loss of HLA-I expression (i.e. Phenotype I) is frequently associated with the absence of functional β2m protein, due to mutations in the β2m gene located in chromosome 15 (region 15q21), followed by a loss of the second copy of this gene caused by LOH in chromosome 15 (LOH-15) (del Campo et al, (2014)). These genetic changes are frequently found during natural cancer progression. Therefore, β2m/HLA-I expression should be considered as a biomarker and a potential therapeutic target in the development of cancer.

Accordingly, if the comparing step c) reveals an increase in texosomes associated with tumour cells of phenotype I this may be indicative of cancer progression.

In another aspect, the present invention relates to a method for determining the prognosis of a subject with cancer, said method comprising:

a) determining the major histocompatibility complex profile of the total tumour burden of said subject with cancer in accordance with the method of the invention; and

b) repeating step a) and comparing the major histocompatibility complex profile of the total tumour burden of said subject with the major histocompatibility complex expression profile of the total tumour burden of said subject determined in step a;

wherein an increase in texosomes associated with tumour cells having aberrant MHC-I expression which is irreversible with cytokines is indicative of poor prognosis.

Alternatively, an increase in the ratio of texosomes associated with tumour cells having aberrant MHC-I expression which is reversible with cytokines to texosomes associated with tumour cells having aberrant MHC-I expression which is irreversible with cytokines is indicative of good prognosis.

In some embodiments, only step a) may be carried out.

For example, where the major histocompatibility complex profile of the total tumour burden of said subject with cancer is determined in step a), and it is found that the number of texosomes associated with tumour cells having aberrant MHC-I expression which is irreversible with cytokines is low, this would be indicative of good prognosis.

In this context “low” may be that less than 20%, or less than 15%, or less than 10%, or less than 5%, or less than 3%, or less than 1% of texosomes in a sample display a phenotype associated with tumour cells having aberrant MHC-I expression which is irreversible with cytokines.

The “level of a texosomes associated with tumour cells having aberrant MHC-I expression which is irreversible with cytokines” may refer to the amount of texosomes in a sample derived from a “hard” lesion by comparison with a control. In this regard, the control may be a peripheral blood cell of the subject or a healthy cell of the same cell type. Suitably, the control in step a) is the same control for step b).

An increase in the number of texosomes on comparison of step b) with step a) refers to any statistically significant increase. Suitably, an increase of at least 5% or at least 10% or at least 20% may be indicative of poor prognosis. However, the skilled person is aware that this will depend on a number of factors including the time period between the samples for step a) and step b). The shorter the time period the more significant an increase of a particular amount is.

Suitably, step b) is conducted at least 1 week, or at least 2 weeks, or at least 3 weeks, or at least a month, or at least 2 months or at least 3 months after step a).

Suitably, step b) may be repeated at subsequent time points to provide an ongoing analysis of cancer progression. For example, the step b) may be repeated on a monthly, 3-monthly or yearly basis.

Suitably, in each step, a plurality of samples (i.e., from different physiological fluids) may be analysed, and/or multiple samples of the same physiological fluid may be analysed, to provide a more representative analysis of the MHC profile of the subject.

Suitably, steps a) and b) are repeated under similar (preferably identical conditions) to minimise variation from the analysis. Suitably, a trend of decreasing texosomes with time (particularly where step b) is repeated one or more times) may be indicative of good prognosis whereas a trend of increasing texosomes may be indicative of poor prognosis.

In another aspect, the present invention relates to a method for determining the therapeutic effect of a medicament used in the treatment of cancer, said method comprising:

a) determining the major histocompatibility complex profile of the total tumour burden of said subject with cancer from a physiological sample of the subject prior to treatment with a medicament in accordance with the method of the invention;

b) determining the major histocompatibility complex profile of the total tumour burden of said subject with cancer, in accordance with the method of step a), from a physiological sample of the subject obtained after treatment with a medicament for a predetermined period; and

c) comparing the major histocompatibility complex profiles obtained in steps a) and b).

In this context, by “prior to treatment” it is meant prior to the treatment which will occur between the analysis in step a) and the analysis in step b). Accordingly, the subject may have already have been treated by the medicament to be tested or another medicament.

Suitably, at the time point of sample a) the subject may not have undergone treatment with the medicament to be tested.

Step b) is undertaken at a time period later than that of a) to provide a therapeutic window to determine the effectiveness of the treatment. A person of ordinary skill in the art is readily aware that different medicaments will need to be administered for different time periods before a therapeutic effect can be determined and that this will be dependent on the medicament, the dosing regime and the type of cancer the subject may have, for example. Accordingly, the skilled person can readily choose a time period between steps a) and b) by which a therapeutic effect may be realised in a subject sensitive to such treatment.

Accordingly, on carrying out the comparison step c), if a therapeutic effect is not found in the comparison of the MHC-I expression profile of steps a) and b) it is indicative that the medicament is not therapeutically effective. For example, where immunotherapy (e.g., cytokine therapy) is administered to the subject an increase in texosomes associated with tumour cells displaying an aberrant MHC-I profile that is not reversible with cytokines, is indicative that the treatment is not therapeutically effective when used as the sole treatment.

Suitably, step b) may be conducted at least 1 week, or at least 2 weeks, or at least 3 weeks, or at least a month, or at least 2 months or at least 3 months after step a).

Suitably, steps b) and c) may be repeated at subsequent time points to provide an ongoing analysis of therapeutic effect of the medicament. For example, the steps b) and c) may be repeated on a monthly, 3-monthly or yearly basis.

Suitably, in each step a plurality of samples (i.e., from different physiological fluids) may be analysed and/or multiple samples of the same physiological fluid may be analysed to provide a more representative analysis of the MHC profile of the subject.

Suitably, steps a) and b) are repeated under similar (preferably identical conditions) to minimise variation from the analysis.

Suitably, a trend of decreasing texosomes with time (particularly where steps b) and c) are repeated one or more times) may be indicative of a good therapeutic effect, whereas a trend of increasing texosomes may be indicative of a poor therapeutic effect.

Predicting responsiveness or sensitivity to certain medicaments based on the MHC profile of a tumour as measured by texosome analysis

It is an object of the invention to determine patient populations that have increased sensitivity to treatment of cancer with specific medications based on the MHC-I profile of a tumour (preferably based on the MHC-I profile of the total tumour burden of the patient population based on texosome analysis).

Tumour cells having an aberrant MHC-I profile may have altered response to medicaments and hence, by analysing the MHC-I profile of subject or patient population, appropriate medicaments with will increase the likelihood of response to treatment for the subject or patient population can be selected, or alternatively, a patient population can be selected having an increased likelihood of response to treatment with a particular medicament based on the MHC-I profiling using texosomes.

Accordingly, in one aspect, the present invention provides the use of an isolated sample of texosomes of a subject with cancer to predict responsiveness or sensitivity to a particular treatment, such as treatment with cytokine therapy or HDAC inhibitors.

As used herein, a subject is “responsive” or “sensitive” to treatment if they respond therapeutically such that the cancer is alleviated or abrogates. This means that the life expectancy of an individual affected with the cancer will be increased, or that one or more of the symptoms of the cancer will be reduced or ameliorated. The term encompasses a reduction in cancerous cell growth or tumour volume. Whether a mammal responds therapeutically can be measured by many methods well known in the art, such as PET imaging.

As used herein “increasing the sensitivity rate” of a medicament refers to selecting a subpopulation that has a statistically significant increase in the number of subjects that are “sensitive” to said treatment compared to the original patient population.

Immunotherapy

The term “immunotherapeutic agent” can include any molecule, peptide, antibody or other agent which can modulate a host immune system in response to an antigen, such as expressed by a tumour or cancer in the subject. Immunotherapeutic strategies include administration of vaccines, antibodies, cytokines, chemokines, as well as small molecular inhibitors, anti-sense oligonucleotides, and gene therapy, as described further below (see, for example, Mocellin et al. (2002) and Dy et al. (2002)).

Immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumour antigen). Immunotherapy can also focus on using the cytotoxic lymphocyte-recognized epitopes of cancer cell lines. Alternatively, antisense polynucleotides, ribozymes, RNA interference molecules, triple helix polynucleotides and the like, can be used to selectively modulate biomolecules that are linked to the initiation, progression, and/or pathology of a tumour or cancer.

In the present invention, an “immunotherapy” refers to any medicament which will lead to increased cytokine production and/or upregulation of MHC-I expression in the subject.

Accordingly, the present invention provides methods of increasing the sensitivity rate of an immunotherapy to treat cancer in a patient population, said method comprising selecting a subpopulation, wherein the major histocompatibility complex expression profile of the total tumour burden has been determined using texosome analysis in the subpopulation, and said subpopulation has:

a) a level below a predetermined threshold of texosomes associated with tumour cells unresponsive to cytokines.

Suitably, the level of texosomes associated with tumour cells unresponsive to cytokines in the sub population may be statistically significantly lower than the level of texosomes associated with tumour cells unresponsive to cytokines in the patient population.

The present invention further provides a method of identifying a subject having increased likelihood of responsiveness or sensitivity to an immunotherapy, said method comprising:

a) determining the level of texosomes associated with tumour cells that are unresponsive to cytokines;

wherein a level below a predetermined threshold is indicative of having an increased likelihood of responsiveness or sensitivity to the immunotherapy.

The present invention also provides a method of identifying a subject having responsiveness or sensitivity to an immunotherapy, said method comprising:

a) determining the level of texosomes associated with tumour cells that are unresponsive to cytokines;

wherein a level below a predetermined threshold is indicative of having responsiveness or sensitivity to the immunotherapy.

Suitably the predetermined threshold of texosomes associated with tumour cells that are unresponsive to cytokines may be less than 25%, or less than 20%, or less than 15%, or less than 10%, or less than 5% of the total texosomes in the sample tested.

The present invention also provides immunotherapy for use in the treatment of cancer in a subject, wherein the subject has undergone texosome MHC-I profile analysis.

The present invention further provides a method of treating cancer in a subject identified as having an increased likelihood of responsiveness or sensitivity to an immunotherapy in accordance with a method of the present invention, comprising:

a) administering a therapeutically effective amount of the immunotherapy.

Preferably, the subject has been identified as a responder. Suitably an immunotherapy in accordance with the invention will typically fall into one of the following two categories:

a) cytokines (e.g. cytokine therapy); or

b) therapies which promote endogenous cytokine production, preferably at the site of the tumour.

Types of cytokine therapy and the uses thereof in accordance with the invention are explained in more detail in the “cytokine therapy” section below.

Therapies which promote endogenous cytokine production are known in the art, and examples include: dendritic cell vaccines, adoptive T cell vaccines, peptide vaccines, checkpoint inhibitors and antibodies which induce the release of cytokines.

In some embodiments, the immunotherapy is cancer cell-specific. In some embodiments, immunotherapy can be “untargeted”, which refers to administration of agents that do not selectively interact with immune system cells, yet modulates immune system function to increase endogenous cytokines. Representative examples of untargeted therapies include, without limitation, chemotherapy, gene therapy, and radiation therapy.

In one embodiment, immunotherapy comprises adoptive cell-based immunotherapies. Well known adoptive cell-based immunotherapeutic modalities, including, without limitation, irradiated autologous or allogeneic tumour cells, tumour lysates or apoptotic tumour cells, antigen-presenting cell-based immunotherapy, dendritic cell-based immunotherapy, adoptive T cell transfer, autologous immune enhancement therapy (AIET), cancer vaccines, and/or antigen presenting cells. Such cell-based immunotherapies can be further modified to express one or more gene products to further modulate immune responses, such as expressing cytokines like GM-CSF, and/or to express tumour-associated antigen (TAA) antigens, such as Mage-1, gp-100, and the like.

In another embodiment, immunotherapy comprises non-cell-based immunotherapies. In one embodiment, compositions comprising antigens with or without vaccine-enhancing adjuvants are used. Such compositions exist in many well-known forms, such as peptide compositions, oncolytic viruses, recombinant antigen comprising fusion proteins, and the like. In still another embodiment, immunomodulatory interleukins, such as IL-2, IL-6, IL-7, IL-12, IL-17, IL-23, and the like, as well as modulators thereof (e.g., blocking antibodies or more potent or longer lasting forms) are used. In yet another embodiment, immunomodulatory cytokines, such as interferons, G-CSF, imiquimod, TNF alpha, and the like, as well as modulators thereof (e.g., blocking antibodies or more potent or longer lasting forms) are used. In another embodiment, immunomodulatory chemokines, such as CCL3, CCL26, and CXCL7, and the like, as well as modulators thereof (e.g., blocking antibodies or more potent or longer lasting forms) are used. In another embodiment, immunomodulatory molecules targeting immunosuppression, such as STAT3 signaling modulators, NFkappaB signaling modulators, and immune checkpoint modulators, are used.

The term “immune checkpoint” refers to a group of molecules on the cell surface of CD4+ and/or CD8+ T cells that fine-tune immune responses by down-modulating or inhibiting an anti-tumour immune response. Immune checkpoint proteins are well known in the art and include, without limitation, CTLA-4, PD-1, VISTA, B7-H2, B7-H3, PD-LI, B7-H4, B7-H6, 2B4, ICOS, HVEM, PD-L2, CD 160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, TIGIT, HHLA2, butyrophilins, and A2aR (see, for example, WO 2012/177624). The term further encompasses biologically active protein fragment, as well as nucleic acids encoding full-length immune checkpoint proteins and biologically active protein fragments thereof. In some embodiment, the term further encompasses any fragment according to homology descriptions provided herein.

“Anti-immune checkpoint therapy” refers to the use of agents (“checkpoint inhibitors”) that inhibit immune checkpoint nucleic acids and/or proteins. Inhibition of one or more immune checkpoints can block or otherwise neutralize inhibitory signalling to thereby upregulate an immune response in order to more efficaciously treat cancer. Exemplary agents useful for inhibiting immune checkpoints include antibodies, small molecules, peptides, peptidomimetics, natural ligands, and derivatives of natural ligands, that can either bind and/or inactivate or inhibit immune checkpoint proteins, or fragments thereof; as well as RNA interference, antisense, nucleic acid aptamers, etc., that can downregulate the expression and/or activity of immune checkpoint nucleic acids, or fragments thereof. Exemplary agents for upregulating an immune response include antibodies against one or more immune checkpoint proteins block the interaction between the proteins and its natural receptor(s); a non-activating form of one or more immune checkpoint proteins (e.g., a dominant negative polypeptide); small molecules or peptides that block the interaction between one or more immune checkpoint proteins and its natural receptor(s); fusion proteins (e.g. the extracellular portion of an immune checkpoint inhibition protein fused to the Fc portion of an antibody or immunoglobulin) that bind to its natural receptor(s); nucleic acid molecules that block immune checkpoint nucleic acid transcription or translation; and the like. Such agents can directly block the interaction between the one or more immune checkpoints and its natural receptor(s) (e.g., antibodies) to prevent inhibitory signalling and upregulate an immune response. Alternatively, agents can indirectly block the interaction between one or more immune checkpoint proteins and its natural receptor(s) to prevent inhibitory signalling and upregulate an immune response. For example, a soluble version of an immune checkpoint protein ligand such as a stabilized extracellular domain can binding to its receptor to indirectly reduce the effective concentration of the receptor to bind to an appropriate ligand. Suitably, anti-PD-1 antibodies, anti-PD-LI antibodies, and anti-CTLA-4 antibodies, either alone or in combination, may be used to inhibit immune checkpoints.

By utilising checkpoint inhibitors, a patient's CTLs will be able to engage with tumour antigens, promoting an immune response. This will lead to increased interferon gamma production and the up-regulation of MHC. Any checkpoint inhibitor which increases endogenous cytokines (preferably at the tumour site) can be used in accordance with the present invention.

Suitably, the immunotherapy may be an antibody (preferable a monoclonal antibody) which induces the release of cytokines in the subject with cancer.

Unless otherwise specified here within, the terms “antibody” and “antibodies” broadly encompass naturally-occurring forms of antibodies (e.g. IgG, IgA, IgM, IgE) and recombinant antibodies such as single-chain antibodies, chimeric and humanized antibodies and multi-specific antibodies, as well as fragments and derivatives of all of the foregoing, which fragments and derivatives have at least an antigenic binding site. Antibody derivatives may comprise a protein or chemical moiety conjugated to an antibody.

The term “antibody” as used herein also includes an “antigen-binding portion” of an antibody (or simply “antibody portion”). The term “antigen-binding portion,” as used herein, refers to one or more fragments of an antibody that retain the ability to specifically bind to an antigen (e.g., a biomarker polypeptide or fragment thereof). It has been shown that the antigen-binding function of an antibody can be performed by fragments of a full-length antibody.

Antibodies may be polyclonal or monoclonal; xenogeneic, allogeneic, or syngeneic; or modified forms thereof (e.g. humanized, chimeric, etc.). Antibodies may also be fully human. Preferably, antibodies of the present invention bind specifically or substantially specifically to a biomarker polypeptide or fragment thereof. The terms “monoclonal antibodies” and “monoclonal antibody composition”, as used herein, refer to a population of antibody polypeptides that contain only one species of an antigen binding site capable of immunoreacting with a particular epitope of an antigen, whereas the term “polyclonal antibodies” and “polyclonal antibody composition” refer to a population of antibody polypeptides that contain multiple species of antigen binding sites capable of interacting with a particular antigen. A monoclonal antibody composition typically displays a single binding affinity for a particular antigen with which it immunoreacts.

Antibodies may also be “humanized”, which is intended to include antibodies made by a non-human cell having variable and constant regions which have been altered to more closely resemble antibodies that would be made by a human cell. For example, by altering the non-human antibody amino acid sequence to incorporate amino acids found in human germline immunoglobulin sequences. The humanized antibodies of the present invention may include amino acid residues not encoded by human germline immunoglobulin sequences (e.g., mutations introduced by random or site-specific mutagenesis in vitro or by somatic mutation in vivo), for example in the CDRs. The term “humanized antibody”, as used herein, also includes antibodies in which CDR sequences derived from the germline of another mammalian species, such as a mouse, have been grafted onto human framework sequences.

Suitably, the immunotherapy may be a dendritic cell vaccine. Adoptive transfer of activated dendritic cells comprises the stimulation of dendritic cells harvested from a patient to activate a cytotoxic response towards the tumour cells. Dendritic cells can be stimulated for example by pulsing them with an antigen or transfecting them with a viral vector. The stimulated dendritic cells are infused back into the patient and initiate a cytotoxic immune response against the tumour cells. Adoptive transfer of tumour infiltrating lymphocytes comprises the in-vitro amplification of lymphocytes that have been isolated from the patient's tumour tissue and have anti-tumour activity. Suitably, dendritic cells which increase the level of cytokines in the subject may be encompassed in the present invention.

Cytokine Therapy

Cytokine therapy has proven to be a novel therapeutic approach in treating patients with advanced malignancies. The purpose of this type of therapy is to manipulate the immune response in such a way as to generate the appropriate immune effector cells to eradicate solid tumours. Cytokine therapy is typically administrated after the conventional form of therapies have been performed such as chemotherapy, radiotherapy, and surgery. Various regimens of cytokine administration have been implemented in eradicating solid tumours in patients with melanoma and renal cell cancer.

Two types of cytokines of interest which are used to treat patients with cancer are interferons (INFs) and interleukins (Ls). There have been clinical trials executed involving the administration of interferon-gamma, interferon-alpha, Interleukin-2, tumour necrosis factor-alpha, and Interleukin-12. INF-alpha, can enhance a patient's immune response to cancer cells by activating certain white blood cells, such as natural killer cells and dendritic cells (Sutlu T and Alici E. 2009). INF-alpha may also inhibit the growth of cancer cells or promote their death. INF-alpha has been approved for the treatment of melanoma, Kaposi sarcoma, and several hematologic cancers.

Like INFs, ILs play important roles in the body's normal immune response and in the immune system's ability to respond to cancer. Researchers have identified more than a dozen distinct ILs, including IL-2, which is also called T-cell growth factor. IL-2 is naturally produced by activated T cells. It increases the proliferation of white blood cells, including cytotoxic T cells and natural killer cells, leading to an enhanced anticancer immune response. IL-2 also facilitates the production of antibodies by B cells to further target cancer cells.

Medicament used for cytokine therapy are a diverse recombinant, synthetic and natural preparations, often cytokines, such as granulocyte colony-stimulating factor (G-CSF), interferons, imiquimod and cellular membrane fractions e.g. from bacteria. Others include IL-2, IL-7, IL-12, various chemokines, synthetic cytosine phosphate-guanosine (CpG), oligodeoxynucleotides and glucans which are currently being investigated extensively in clinical and preclinical studies.

Cytokine therapy may up-regulate HLA-I expression to present tumour antigens (including newly released peptides) to enable recognition by cytotoxic T lymphocytes (CTLs). Clearly, such HLA-I up-regulation may not be possible for malignant cells containing irreversible (i.e. hard) structural alterations in HLA genes, or that are resistant to cytokine-mediated up-regulation due to defects in signal transduction pathways. Hence, tumour cells harbouring hard structural MHC aberrations may escape immune recognition even after cytokine therapy and so would be resistant to cytokine therapy.

Accordingly, the present invention provides methods of increasing the sensitivity rate of a cytokine therapy to treat cancer in a patient population, said method comprising selecting a subpopulation, wherein the major histocompatibility complex expression profile of the total tumour burden has been determined using texosome analysis in the subpopulation and said subpopulation has:

a) a level below a predetermined threshold of texosomes associated with tumour cells unresponsive to cytokines.

Suitably, the level of texosomes associated with tumour cells unresponsive to cytokines in the sub population may be statistically significantly lower than the level of texosomes associated with tumour cells unresponsive to cytokines in the patient population.

The present invention further provides a method of identifying a subject having increased likelihood of responsiveness or sensitivity to cytokine therapy, said method comprising:

a) determining the level of texosomes associated with tumour cells that are unresponsive to cytokines;

wherein a level below a predetermined threshold is indicative of having an increased likelihood of responsiveness or sensitivity to cytokine therapy.

The present invention also provides a method of identifying a subject having responsiveness or sensitivity to cytokine therapy, said method comprising:

a) determining the level of texosomes associated with tumour cells that are unresponsive to cytokines;

wherein a level below a predetermined threshold is indicative of having responsiveness or sensitivity to cytokine therapy.

Suitably the predetermined threshold of texosomes associated with tumour cells that are unresponsive to cytokines may be less than 25%, or less than 20%, or less than 15%, or less than 10%, or less than 5% of the total texosomes in the sample tested.

The present invention also provides cytokine therapy for use in the treatment of cancer is a subject, wherein the subject has undergone texosome MHC-I profile analysis.

The present invention further provides a method of treating cancer in a subject identified as having an increased likelihood of responsiveness or sensitivity to cytokine therapy in accordance with a method of the present invention, comprising:

a) administering a therapeutically effective amount of a cytokine.

Preferably, the subject has been identified as a responder.

Cancer Vaccines

Certain peptides and peptide treated dendritic cells can be used as part of anti-cancer vaccinations. This harnesses the ability of cytotoxic T lymphocytes (CTLs) to attack and destroy cells. They are produced through differentiation in response to antigen presentation. Precursor cells of the immune system are presented antigens as part of the Class I MHC system (human leukocyte antigen (HLA) in humans) and cancer cell antigen systems, and will differentiate accordingly. Short peptides can bind to Class I MHC molecules and induce T cells to differentiate and consequently attack and destroy cancer cells expressing the complex of the MHC class I antigen and the cancer antigen (Slingluff 2011, US 2016367649). Researchers are developing antigen proteins of various cancers, to develop into vaccinations. Cancer treatment vaccinations have received regulatory approval, for example sipuleucel-T (Provenge®) is approved for use in men with metastatic prostate cancer and talimogene laherparepvec (T-VEC, or Imlygic®) for the treatment of some patients with metastatic melanoma.

Loss of a single HLA-I allele can be sufficient to impair the detection and killing of tumour cells by CTLs, and results in a tumour variant which can evade immune surveillance (Carretero et al, (2008)). For example, loss of HLA-A2 expression in a tumour could compromise the efficacy of immunotherapy as it has one of the widest peptide repertoires among human MHC-I molecules and many cancer vaccines use HLA-A2-specific tumour peptides to stimulate HLA-A2-restricted CTL responses (Nishimura et al, 2015).

Accordingly, the present invention provides a method of increasing the sensitivity rate of a cancer vaccines utilising HLA-A2 specific peptides to treat or prevent cancer in a patient population, said method comprising selecting a subpopulation, wherein the major histocompatibility complex profile of the total tumour burden has been determined using texosome analysis in the subpopulation, and said subpopulation has:

a) a level below a predetermined threshold of texosomes associated with tumour cells having a loss of HLA-A2 expression.

Suitably, the level of texosomes associated with tumour cells having a loss of HLA-A2 expression in the sub population may be statistically significantly lower than the level of texosomes associated with tumour cells having a loss of HLA-A2 expression in the patient population.

Further, the present invention provides a method of identifying a subject having increased likelihood of responsiveness or sensitivity to a cancer vaccine utilising a HLA-A2 specific peptide, said method comprising:

a) determining the level of texosomes associated with tumour cells having a loss of HLA-A2 expression;

wherein a level below a predetermined threshold is indicative of having an increased likelihood of responsiveness or sensitivity to a cancer vaccine utilising a HLA-A2 specific peptide.

The present invention also provides a method of identifying a subject having responsiveness or sensitivity to a cancer vaccine utilising a HLA-A2 specific peptide, said method comprising:

a) determining the level of texosomes associated with tumour cells having a loss of HLA-A2 expression;

wherein a level below a predetermined threshold is indicative of having responsiveness or sensitivity to a cancer vaccine utilising a HLA-A2 specific peptide.

Suitably the predetermined threshold of texosomes associated with tumour cells having a loss of HLA-A2 expression may be less than 25%, or less than 20%, or less than 15%, or less than 10%, or less than 5% of the total texosomes in the sample tested.

The present invention also provides a cancer vaccine for use in the treatment of cancer is a subject, wherein the subject has undergone texosome MHC-I profile analysis. Suitably, the cancer vaccine may utilise a HLA-A2 specific peptide.

HDAC Inhibitors

Inhibitors of HDACs modulate transcription and induce cell growth arrest, differentiation, and apoptosis. HDAC inhibitors (HDACIs) also enhance the cytotoxic effects of therapeutic agents used in cancer treatment, including radiation and chemotherapeutic drugs.

The term “HDAC” refers to a family of enzymes that remove acetyl groups from a protein, for example, the ε amino groups of lysine residues at the N-terminus of a histone. The HDAC can be a human HDAC, including, HDAC1, HDAC2, HDAC3, HDAC4, HDAC5, HDAC6, HDAC7, HDAC8, HDAC9, HDAC10, and HDAC11. The HDAC also can be derived from a protozoal or fungal source.

HDAC inhibitors (HDACIs) typically contain three structural elements which are analogous to the structure of acetyllysine. These three structural elements are a zinc binding group (M), which is responsible for chelation of zinc in the active site, a linker region (L), which binds to the hydrophobic channel that connects the active site to the outer enzyme surface, and a capping group (Cap), which interacts with residues at the outer enzyme surface.

Examples of HDAC inhibitors include: SAHA, Romidepsin, Valproic Acid, PCI-24781, ITF-2357, MS275, Panbinoastat, Belinostat, Vorinostat, MGCD0103 and EVP-0334.

HDAC inhibitors have been approved for or clinical trials are underway in at least the following: T-cell lymphoma, multiple myeloma, renal cancer, Hodgkins lymphoma, Follicular lymphoma, leukemia, acute myeloid leukemia, melanoma, non-small cell lung cancer, solid tumours, prostate cancer, diffuse large B-cell lymphoma and mesothelioma, for example.

The present invention provides HDAC inhibitors for use in the treatment or prevention of cancer is a subject, wherein the subject has undergone texosome MHC-I expression profile analysis.

HLA-G

HLA-G is an MHC class I molecule which is involved in the immune tolerance. Whilst HLA-G expression is restricted in normal tissues, it is broadly expressed in malignant tumours and has been detected in more than 1000 tumour lesions—see Wastowski et al. (The American Journal of Pathology, Vol. 182, No.2, February 2013). Wastowski et al. confirm that HLA-G can be incorporated into tumour-derived exosomes and HLA-G aberrant expression has been suggested to be part of the strategies that tumours use to escape from the host's immunosurveillance with HLA-G mediated protection of tumour cells against NK and T-cell cytotoxicity having been demonstrated in vitro.

Furthermore, a correlation between HLA_G expression and poor clinical outcome has been demonstrated in various tumours including: melanoma, B cell chronic lymphocytic leukaemia, nasopharyngeal carcinoma, breast cancer, esophageal squamous cell carcinoma, non-small cell lung cancer, colorectal tumours and neuroblastoma.

As explained earlier, texosomes (e.g. exosomes) secreted by tumour cells are small MHC-bearing vesicles of endocytic origin which are released into the extracellular milieu (Miller and Grunewald, 2015). It is postulated that the HLA-G profile comes from the tumour cells from which they are derived from because exosomes do not contain the machinery to manufacture proteins. Without wishing to be bound by theory, it is believed that the HLA-G profile that comes along with the membrane when a texosome is formed are representative of the tumour cells they come from i.e. that the same altered phenotypes are present on the texosomes and at the same ratios as they were found on the parent tumour cells.

Accordingly, the present invention provides methods for determining the HLA-G expression profile of a tumour using texosome analysis.

In one aspect, the present invention relates to a method of determining the HLA-G expression profile of a tumour of a subject with cancer comprising the steps of:

a) obtaining a physiological fluid sample comprising texosomes or a texosome-enriched sample of extracellular vesicles from a physiological fluid sample of the subject;

b) identifying the phenotypes of texosomes associated with tumour cells having HLA-G expression.

Suitably, the method may determine the HLA-G expression profile of the total tumour burden of a subject.

Suitably, the step identifying the phenotypes of texosomes associated with tumour cells having HLA-G expression, identifies tumour cells having aberrant HLA-G expression.

As used herein “tumour cells having normal HLA-G expression” refers to tumour cells having HLA-G which substantially corresponds to HLA-G expression of a normal or otherwise healthy cell of a peripheral blood cell or a cell of the same type as the tumour cell. For example, where the tumour cell is found in the lung a normal HLA-G expression profile may be one that substantially corresponds to that of a healthy lung cell. Thus, the HLA-G profile of the tumour cell may be compared to a peripheral blood cell and/or a healthy cell of the same type to determine if the tumour cell has a normal HLA-G profile. By “substantially corresponds” in this context it is meant that the HLA-G expression of the tumour cells is within the normal or physiologically normal variability seen between healthy cells of the same type. Typically, normal or healthy blood cells may not express HLA-G.

As used herein “tumour cells having an aberrant HLA-G expression” refers to tumour cells which do not have a normal HLA-G expression compared to a normal or otherwise healthy or physiologically normal cell of a peripheral blood cell or a cell of the same type as the tumour cell. Typically, aberrant tumour cells have an up regulation of HLA-G expression. Suitably, the up regulation may be at least 5% or at least 10% or at least 15% or at least 20% or at least 25% of the expression of a normal or otherwise healthy cell of a peripheral blood cell or a cell of the same type as the tumour cell.

As HLA-G expression of tumour cells has been correlated with poor clinical outcome, texosome analysis for HLA-G expression can be used to for determining the prognosis of a subject with cancer.

Hence, the present invention provides a method for determining the prognosis of a subject with cancer by determining the HLA-G profile of a tumour (preferably the total tumour burden of the subject) with cancer in accordance with the method disclosed herein. Suitably, the determining step may be repeated one or more times and the results compared to determine prognosis.

The general techniques and methodologies for determining the MHC profile are applicable to determine HLA-G profile, including the methodologies for isolating texosomes, types of cancer, subject to be treatment and time periods between analysis etc.

### EXAMPLES

Preparation of Extracellular Vesicles

In the proposed examples of the method of the invention a sample of blood is obtained from the subject and centrifuged (500×g for 20 minutes at 10° C.) to pellet cells. The cells are discarded and remaining cellular debris removed from the supernatant by further centrifugation (16,000×g for 20 minutes at 10° C.). The supernatant is then filtered (0.22 μM) and the extracellular vesicles remaining in the filtrate pelleted by ultracentrifugation (110,000×g for 70 minutes at 10° C.) (Beckman Ti70 rotor). The extracellular vesicles fraction may be further purified by ultracentrifugation in a 20 and 40% sucrose gradient before being washed with filter sterilised phosphate-buffered saline (PBS).

Immunocytometric Analysis of Texosomes

Method 1

Paramagnetic beads ready-coated with antibodies to multiple tumour-specific antigens are added to a suspension of the extracellular vesicles fraction at a limiting bead to extracellular vesicle ratio. For this purpose beads of the minimum size within the range of the detectors of the flow cytometer are used. A ratio approaching the idealised ratio of 1:1 is desired to facilitate discrimination between sub-populations of texosomes with different major histocompatibility complex phenotypes. The suspension is incubated for 24 hours on a rolling machine at room temperature before aliquots of the suspension of texosome-bead complexes are incubated in a final volume of 100 μL containing 0.5% bovine serum albumin (BSA) in phosphate-buffered saline (PBS) and 10 μL of fluorescently labelled antibodies. After incubation for one hour at room temperature, the beads are washed and analysed by flow cytometry. For each aliquot different combinations and concentrations of fluorescently labelled antibodies are used to facilitate the statistical determination of the major histocompatibility complex expression profile of the texosomes.

Method 2

The texosome fraction is isolated from the extracellular vesicles fraction using affinity chromatography. Immobilised antibodies to multiple tumour-specific antigens are employed to minimise bias and the inadvertent preferential isolation of subpopulations of the texosome fraction. Isotopically labelled antibodies are then added to aliquots of a suspension of the enriched texosome fraction and incubated in phosphate-buffered saline (PBS) for one hour at room temperature. For each aliquot different combinations and concentrations of isotopically labelled antibodies are used to facilitate determination of the major histocompatibility complex expression profile of the texosomes. Each aliquot is then analysed by mass cytometry and the major histocompatibility complex expression profile determined statistically.

Tumour Cell Culture

Human tumour cell lines are grown in an incubator at 37° C. and 5% CO2 in a culture media (RPMI and Dulbecco) supplemented with 10% fetal bovine serum, L-glut and antibiotics. Cells are analyzed for expression of HLA-I using a wide panel of antibodies directed against HLA-I specificities, β2m and APM (antigen presentation machinery) components using flow cytometry, PCR and gene sequencing.

Flow Cytometry

The cell surface expression of HLA class I molecules are evaluated by indirect immunofluorescence using various antibodies and fluorochromes.

Protein Extraction and Western Blotting

Protein extract is obtained from exosomes and from cultured tumour cells using RIPA buffer (Sigma) with protease inhibitors (Sigma). Proteins are separated by 15% SDS-PAGE according to Laemmli, and transferred to a nitrocellulose membrane. Membranes are incubated overnight with polyclonal rabbit anti-b2m antibodies (diluted 1:2000), one hour with AP-conjugated secondary antibody (Bio-Rad Labs, USA), and developed with the AP-Conjugate Substrate Kit (Bio-Rad Labs,USA). Anti-alpha-tubulin antibodies (Sigma) are used to confirm that similar amounts of proteins were loaded from each extract.

Extraction of genomic DNA from peripheral blood cells and genomic HLA typing

The extraction of DNA from blood samples is done using the Qiagen DNA isolation kit and for RNA isolation RNeasy Mini Kit, Qiagen is used.

Low resolution genomic typing is performed by analyzing specific nucleotide (SSO) using the Dynal Rel. SSO HLA-A, B, C, DR and DQ kit. The results are interpreted with the pattern matching program Dynal 5.04. In some cases for high-resolution typing by sequencing of individual loci by SBT (Sequence-based typing) is used. This amplification is performed by PCR using the kit Allele SEQR Atria Genetics for locus-specific HLA-A, B and C. The sequencing reactions are done with “big-dye terminators”, and subjected to electrophoresis in the automated sequencer 3130xl Genetic Analyzer (Applied Biosystem HITACHI). The results are analyzed using the Assign 3.5 (Conexio Genomics) software.

The obtained cDNA from tumour cells and exosome material using quantitative real time PCR is used to study the expression of HLA-A, HLA-B, HLA-C, b2m genes, as well as molecules involved in antigen processing and presentation (APM) e.g. (TAP1, TAP2, LMP2, LMP7, tapasin and calnexin). The DNA is also used for studies of structural alterations, such as loss of heterogeneicity (LOH) and mutations (gene sequencing).

RNA isolation, reverse transcription and quantitative real time-PCR

Total RNA is extracted from culture cell lines and exosome suspensions using the RNeasy® Mini Kit (QIAGEN, Germany) cDNA synthesis is performed with the iSriptTMcDNA Synthesis Kit(BIO-RAD laboratories, California) and analyzed for b2m gene expression by quantitative real-time PCR (qRT-PCR). To control for variations in amounts of mRNA, the glucose-6-phosphate dehydrogenase (G6PDH) gene is amplified as well. All reactions are performed in a LightCycler instrument (Roche) using the primers provided in the Housekeeping Gene Set Kit (Roche Diagnostics). The final expression levels of target genes are given relative to the expression levels of G6PDH.

RT-PCR is conducted to analyze the gene expression level by evaluating the mRNA copy number of the gene of interest in the studied samples. This is done specifically for a given amplification using specific probes or a non-specific detection of a double-stranded DNA using the SGRB-green I probe is used. In the case of non-specific detection, for confirmation of the specificity of the amplification, melting curves are performed and checked on agarose gel. The RT-PCR is done using a Light-Cycler. To measure the transcription of the HLA-I heavy chain we use the kit LC-Fast Start DNA Master Hybridization Probes. The following oligonucleotides are used as probes: CCATCCGCTGCCAGGTCA (bound to fluorescein) and TGTGATCTCCGCAGGGTAGAAGC (linked to the chromogen Red640). For the locus-specific transcription analysis we use Taq-man (Applied Biosistems) probes for Locus A (ref. HS00740413_g1), for locus B (ref. HS00818803_g1), and for locus C (ref. HS00740298_g1); G6PDH y de HPRT will be used as housekeeping genes. Their expression is used as reference genes for the studied ones.

Genomic sequencing of cDNA and DNA

We use specific primers for PCR amplification of the DNA fragments to be sequenced (for HLA alleles and b2m). The PCR products are purified using Amicon columns (Microcon 100; Millipore). Sequencing reaction is done with the kit Big Dye Terminator Cycle Sequencing for automatic sequencer ABI-PRISM 310 (PE Applied Biosystem). The product of the reaction is purified on the Centri-sep columns (Princenton Separation), rehydrated with the STR (Template Suppression Reagent) and analyzed by the sequencer. Data analysis is performed by means of the Software-PE Applied Biosystem.

Microsatellite analysis for the detection of the loss of heterozygosity (LOH)

Eight short tandem repeat (STRs) markers (7 en 6p21 y 1 en 6q21) are used for the LOH study on chromosome 6 (HLA heavy chain genes), and five markers—on chromosome 15 (b2m gene). In some cases when the haplotype is not clearly determined additional markers corresponding to the HLA class I region are used. The amplification reaction is done in 15 mkl volume using 1,5 mkl of DNA and 1 mkl of the primer mixture (5 mkM of each). The products of the amplification are analysed by 5% acrylamide gel electrophoresis and sequenced using an automatic sequencer-ABI PRISM 377 ADN (PE Applied Biosystem). Data analysis is performed using the software Genotyper programme (PE Applied Biosystem). The results obtained in exosomes and in tumours/metastasis are compared with those obtained on PBLs. Results obtained in microdissected tumour samples are compared with those obtained in stroma samples. LOH is calculated as: Height of tumour allele two/height of area of tumour allele one divided by the Height of normal allele two/height of area of normal allele one. LOH is assigned when more than 25% of signal reduction of one allele was observed in the tumour simples as compared with the control PBL sample.

Analysis of HLA-I in Tumour Specimens

Study of the HLA class I expression in tumours using immunohistochemistry.

We use biotin-streptavidine immunohistological technique with peroxidase developing system (Kit Super-sensitive multilink-HRO/DAB (Biogenex)). We use a panel of monomorphic locus-specific antibodies that define polymorphic epitopes (suggested by the 12th International Histocompatibility Workshop, described in Cabrera et al, 2000). To analyze allelic expression we do HLA class I genomic typing. The normal tissue present in the tumour section is used to control that the antibody works using this technique.

Microdissection of Frozen Tissue Sections

Cryopreserved 4-8 μm thick sections of tumour specimens are normally fixed in 70% ethanol for 10 min, stained with a 0.05% wt/vol solution of toluidine blue for 30 sec to distinguish the tumour cells from the stroma, and dehydrated using different ethanol concentrations (50%, 75%, 95% and 100%). These are later used for tumour cells isolation using laser microdissection (ZEISS Microlaser Systems Protocols) and their collection into Beckman tubes with silicone membranes (PALM Adhesive Caps). DNA from microdissected tumour tissues was obtained using the Qiagen tissue DNA isolation kit (Qiagen, Leiden, Netherlands).

Extraction of DNA from microdissected tissue samples

The tube that contains the fragments is immediately filled to 100 ml with the digestion buffer (Taq pol buffer 1×, 0.1 mg/ml of proteinase K and 1% Tween 20) and incubated at 56° C. for 45 minutes. Then the proteinase K is inactivated by incubation for 15 minutes at 95° C. The product of the digestion is aliquoted and frozen at −40° C. The extraction of DNA is done using a kit for microextraction.

Synthesis and Specific Amplification of cDNA

RT-PCR is done using the commercial kit—Reverse Transcription System, Promega. Specific primers for HLA class I molecules and β2m are used with control amplification of beta-actin. PCR products are analysed by electrophoresis on 1% agarose gels.

Statistical Methods

The Wilcoxon rank-sum test was used to identify differences in immunohistochemical staining scores between tumor epithelium and normal layers (surface and deep) of the urothelium for individual APM components. Clinicopathologic information including tumor histology (three groups: urothelial, squamous and mixed/other), UC grade (three groups: 1-2, 3 and 4), pathologic stage (two groups: low, including T1-2, NO, MO and high, including T3-4 or N1 or M1), and survival were examined with respect to potential associations with immunohistochemical staining score. Wilcoxon rank-sum and Kruskal-Wallis tests were used for univariate two- and three-group comparisons, respectively, for histology, grade and stage. Individual Cox proportional hazards models were fit for survival data using two approaches: (1) including all cancers and adjusting for age, stage, histology, grade, and use of neoadjuvant or adjuvant therapy, and (2) including UC only and adjusting for age, stage, grade and use of neoadjuvant or adjuvant therapy. Hazard ratios estimates, 95% confidence intervals and P values were obtained from these models. All tests were performed with α=0.05.

Quantitative Real-Time RT-PCR Analysis

RNAs were withdrawn using RNeasy Mini Kit (Qiagen, Courtaboeuf, France) according to the manufacturer's protocol. A 1 mg quantity of total RNA from each tumor sample was reverse transcribed at 42 C for 1 hour using the Promega RT system (Promega, Charbonnieres, France). Total RNA from cell lines was reverse transcribed using oligo-(dT) 12-18 priming and M-MLV Reverse Transcriptase (Invitrogen, Cergy Pontoise, France). Real-time RT-PCR was performed in triplicate as previously described with ABI Prism 7000 SDS (Applied Biosystems, Courtaboeuf, France) in a duplex PCR with 40 amplification rounds, using TaqMan Universal PCR Mix, predeveloped TaqMan assay reagent glyceraldehyde-3-phosphate dehydrogenase (GAPDH) as an endogenous control, and an HLA-G specific probe located in exon 5 that targets all HLA-G mRNAs. Quantification was performed relative to amounts of HLA-G transcripts in HLA-G positive JEG-3 using the comparative CT method 33: There were analysed at least two independent experiments comprising treated with cytokines e.g. interferon, and untreated samples.

Statistical Analysis

We used Prism version 5.0 (GraphPad Software, San Diego, Calif.) and the nonparametric U-test for comparison of values obtained by real-time PCR analysis. Data are presented as means SEM for at least three independent experiments. Survival analysis was performed using the log-rank test and graphical representation was performed by Kaplan-Meier. The analysis took into account patients who died of the tumour excluding other reasons (data were validated from the hospital charts and death certificate). Significance was defined as a P <0.05 at a confidence interval of 95%.

### EXAMPLE 1

Texosomes from cell culture supernatants of different cell lines with characterised HLA-I phenotypes and genotypes are characterised. The cell lines utilised include melanoma, prostate, bladder and lung cancer.

Cell lines with characterized “hard” H LA-I alterations (such as mutations in β2m gene of LOH in chromosomes 6 and 15) are used to correlate the HLA-I expression patterns in texosomes with the corresponding parent tumour cell.

Immunodection by flow cytometry is used using a wide panel of anti-HLA and anti-β2m antibodies. The results from the immunodetection are confirmed by western blotting, ELISA or immunoprecipitation.

DNA from the texosomes is used for molecular analysis. HLA-I sequence based typing (SBT) is used to define HLA-I haplotype.

Using thee specific methodology detailed above, HLA-I expression on texosomes (such as tumour derived exosomes) are shown to correlate to the tumour cell lines from which they are derived.

### EXAMPLE 2

Serial blood samples are provided from cancer patients diagnosed with either a liquid (N=X) or solid (N=Y) tumour.

Optimised texosome isolation methods are utilised to prepare enriched samples of texosomes from the blood samples.

Fresh and frozen texosomes are analysed to show comparability.

Autologous peripheral blood cells are used as controls.

Texosome characterisation is conducted using immunodetection by flow cytometry using wide panel of anti-HLA class I and anti-β2m antibodies selected from Part 1, for paired-wise analysis of H LA-I alterations in texosomes and tumour cell samples and the following methods are used to characterize HLA-I expression patterns and alterations:

RT-PCR using primers for HLA-I, β2m and molecules of antigen processing machinery (e.g. tapasin, TAPs, LMPs, etc.).

Microsatellite analysis using several short tandem markers selected for LOH analysis on chromosome 6 spanning the HLA region and 2 markers on chromosome 15 near the β2m gene, as well as searching for alterations in genes encoding antigen processing machinery proteins.

Methylation analysis to identify possible alterations in regulatory mechanism in tumours with HLA heavy chain downregulation.

HLA-I expression on texosomes are shown to correlates with the subject's tumour cell's (either circulating tumour cells or biopsies.

### EXAMPLE 3

A pilot study in patients having either haematological or solid tumours is conducted to compare texosomes to either circulating tumour cells or tumour biopsies.

Serial blood sample of cancer patients with haematological tumours (including Hodgkin's lymphoma or Non-Hodgkin's lymphoma) and solid tumours (including melanoma are collected.

Baseline samples are collected prior to cancer treatment. Serial samples are collected during and after treatment.

Methods for isolating texosomes as described above are used as well as the method for assessing HLA-I alterations in texosomes and tumour cell samples.

Peripheral blood cell samples are collected for HLA typing and used as controls.

HLA-I profile in texosomes at baseline versus post treatment and clinical response to therapy are correlated.

Correlation of HLA-I alterations found in texosomes post treatment to emergence of metastatic progression is undertaken. Also paired-wise comparison of texosomes to tumour cell or tumour biopsies is undertaken.

It is demonstrated that texosome HLA-I alterations can be used as:

prognostic markers; and

diagnostic markers of disease progression.

It is shown that HLA-I phenotype of tumour cells correlate with circulating texosomes.

Although the invention has been described with reference to embodiments or examples it should be appreciated that variations and modifications may be made to these embodiments or examples without departing from the scope of the invention. Where known equivalents exist to specific elements, features or integers of the embodiments or examples, such equivalents are incorporated as if specifically referred to in this specification. In particular, elements of the embodiments or examples, such as the antibodies and immunocytometric methods to be used, that are elements disclosed in and selected from the referenced publications are within the scope of the invention unless specifically disclaimed. The advantages provided by the invention and discussed in the description may be provided in the alternative or in combination in these different embodiments or examples of the invention.

### REFERENCED PUBLICATIONS

Aptsiauri et al (2013) MHC Class I antigens in malignant cells: Immune escape and response to immunotherapy, SpringerBriefs in Cancer Research 6, DOI 10.1007/978-1-4614-6543-0_2.

Cabrera et al (2007) HLA class I expression in metastatic melanoma correlates with tumor development during autologouse vaccination Cancer Immunol Immunother, 56, 709-717.

Carreter et al (2008) Analysis of HLA class I expression in progressing and regressing metastatic melanoma lesions after immunotherapy. Immunogenetics, 60(8), 439-47.

Del Campo et al (2014) Adenovirus expressing β2-microglobulin recovers HLA class I expression and antitumor immunity by increasing T-cell recognition. Cancer Gene Therapy, 21(8), 317-32.

Dy et al. (2002) Novel Targets for Lung Cancer Therapy: Part I Journal of Clinical Oncology. 20, 2881-2894.

Garcia-Lora et al (2003) MHC Class I Antigens, Immune Surveillance, and Tumor Immune Escape J. Cell. Physiol. 193, 346-355.

Garrido et al (1997) Implications for immunosurveillance of altered HLA class I phenotypes in human tumours. Immunol Today, 18(2), 89-95.

Garrido et al (2010) “Hard” and “soft” lesions underlying the HLA class 1 alterations in cancer cells: implications for immunotherapy Int. J. Cancer, 127,249-256.

Garrido et al (2016) MHC class I antigens, immune surveillance, and tumor immune escape. Journal of Cell Physiology, 195(3), 346-55.

Miller, I. V. and Grunewald, T. G. P (2015) Tumour-derived exosomes: Tiny envelopes for big stories Biology of the Cell, 107(9), 287-305.

Mocellin et al. (2002) Adjuvant immunotherapy for solid tumors: from promise to clinical application Cancer Immunology, Immunotherapy, 51(11), 583-595.

Nishimura et al (2015) Cancer immunotherapy using novel tumor-associated antigenic peptides identified by genome-wide cDNA microarray analyses Cancer Science, 106(5), 505-511.

Slingluff (2011), The Present and Future of Peptide Vaccines for Cancer: Single or Multiple, Long or Short, Alone or in Combination? Cancer J., 17(5), 343-350.

Sutlu T, Alici E. Natural killer cell-based immunotherapy in cancer: current insights and future prospects. Journal of Internal Medicine 2009;266(2):154-181.

Wastowski et al. (2013) Human Leukocyte Antigen-G is frequently expressed in gliobastoma and may be induced in vitro by combined 5-Aza-2′-deoxycytidine and interferon-y treatments The American Journal of Pathology, Vol. 182, No.2, February 2013

1) A method of determining the major histocompatibility complex profile of a tumour subject with cancer comprising the steps of:
a) obtaining a physiological fluid sample comprising texosomes or a texosome-enriched sample of extracellular vesicles from a physiological fluid sample of the subject;
b) identifying the phenotype of texosomes associated with tumour cells having aberrant MHC-I expression to determine the major histocompatibility complex profile of the tumour of the subject.

2) The method according to claim 1, wherein the method determines the MHC-I expression profile of the total tumour burden of the subject.

3) The method according to claim 1 or claim 2, wherein the method further comprises identifying the phenotype of texosomes associated with tumour cells that have normal MHC-I expression.

4) The method of any one of claims 1 to 3, wherein the step of identifying the phenotype of texosomes associated with tumours cell having aberrant MHC-I expression determines the level of:
a) texosomes associated with tumour cells having aberrant MHC-I expression which is reversible with cytokines; and/or
b) texosomes associated with tumour cells having aberrant MHC-I expression which is irreversible with cytokines.

5) The method of any one of claims 1 to 4, wherein the subject is a human subject.

6) The method of claim 5, wherein the step of identifying the phenotype of texosomes associated with tumours cells having aberrant MHC-I (HLA-I) expression determines the level of texosomes associated with tumour cells having one or more of the following phenotypes:
Phenotype I—total loss of HLA class I expression;
Phenotype II—loss of HLA class I haplotype;
Phenotype III—Loss of an HLA class I locus;
Phenotype IV—HLA class I allelic loss;
Phenotype V—a compound phenotype;
Phenotype VI—unresponsive to interferons; and
Phenotype VII—Downregulation of classical HLA molecules with aberrant expression of non-classical HLA molecules.

7) The method of any one of claims 1 to 6, wherein the physiological fluid sample is selected from the group consisting of: a blood sample, ascites, pleural effusion, cerebrospinal fluid, and urine.

8) The method of any one of claims 1 to 7, wherein the identifying step comprises the use of exome sequencing.

9) The method of any one of claims 1 to 8, wherein the identifying step comprises the use of flow cytometry.

10) The method of any one of claims 1 to 9, wherein the identifying step comprises the use of mass cytometry.

11) The method of any one of the preceding claims, wherein the method comprises comparing the MHC-I profile of the subject's peripheral blood cells to the MHC-I profile of the texosomes associated with an aberrant MHC-I profile.

12) The method of claim 11, wherein the comparison identifies one or more of the following:
a) differences in locus expression;
b) differences in allelic expression;
c) B2m gene alteration; and
d) loss of heterozygosity in chromosome 6 and/or chromosome 15.

13) The method of any one of the preceding claims, wherein the determining step comprises using a panel of antibodies directed against different loci and allelic specificities.

14) The method of any one of the preceding claims wherein expression of one or more of the following on the texosomes is also measured: TAP1, TAP2, tapasin, calnexin, calreticulin, LMP2 and LMP7.

15) The method of any one of the preceding claims, wherein expression of HLA-G on texosomes is also measured.

16) The method according to any one of the preceding claims, wherein the texosomes are exosomes.

17) The method according to any one of the preceding claims, wherein the subject has a solid tumour.

18) The method according to any one of the preceding claims, wherein the subject has a haematological tumour.

19) The method according to any one of the preceding claims, wherein the subject has a cancer selected from the group consisting of; leukemia, brain cancer, prostate cancer, liver cancer, ovarian cancer, stomach cancer, colorectal cancer, throat cancer, breast cancer, skin cancer, melanoma, lung cancer, sarcoma, cervical cancer, testicular cancer, bladder cancer, endocrine cancer, endometrial cancer, esophageal cancer, glioma, lymphoma, neuroblastoma, osteosarcoma, pancreatic cancer, pituitary cancer, renal cancer, nasopharyngeal cancer.

20) A method for monitoring cancer progression in a subject, said method comprising:
a) determining the major histocompatibility complex profile of the total tumour burden of said subject with cancer in accordance with the method of any one of claims 1 to 19; and
b) repeating step a) and comparing the major histocompatibility complex profile of the total tumour burden of said subject with the major histocompatibility complex profile of the total tumour burden of said subject determined in step a).

21) A method for determining the prognosis of a subject with cancer, said method comprising:
a) determining the major histocompatibility complex profile of the total tumour burden of said subject with cancer in accordance with the method of any one of claims 1 to 19; and
b) repeating step a) and comparing the major histocompatibility complex profile of the total tumour burden of said subject with the major histocompatibility complex expression profile of the total tumour burden of said subject determined in step a;
wherein an increase in texosomes associated with tumour cells having aberrant MHC-I expression which is irreversible with cytokines is indicative of poor prognosis.

22) A method for determining the therapeutic effect of a medicament used in the treatment of cancer, said method comprising:
a) determining the major histocompatibility complex profile of the total tumour burden of said subject with cancer from a physiological sample of the subject prior to treatment with a medicament in accordance with the method of any one of claims 1 to 19;
b) determining the major histocompatibility complex profile of the total tumour burden of said subject with cancer, in accordance with the method of step a), from a physiological sample of the subject obtained after treatment with a medicament for a predetermined period; and
c) comparing the major histocompatibility complex profiles obtained in steps a) and b).

23) Use of an isolated sample of texosomes of a subject with cancer to predict responsiveness or sensitivity of said subject to treatment with an immunotherapy or HDAC inhibitors.

24) Use according to claim 23, wherein the MHC class I expression profile of texosomes isolated from a physiological fluid of the subject is measured.

25) Use according to claim 23 or claim 24, wherein the level of texosomes associated with tumour cells that are unresponsive to interferon is used to predict responsiveness to the immunotherapy.

26) Use according to any one claims 23 to 25, wherein the immunotherapy is selected from the group consisting of: cytokine therapy, dendritic cell vaccines, adoptive T cell vaccines, peptide vaccines, checkpoint inhibitors and antibodies which induce the release of cytokines.

27) A method of increasing the sensitivity rate of an immunotherapy to treat cancer in a patient population, said method comprising selecting a subpopulation, wherein the major histocompatibility complex expression profile of the total tumour burden has been determined using texosome analysis in the subpopulation and said subpopulation has:
a) a level below a predetermined threshold of texosomes associated with tumour cells unresponsive to cytokines.

28) A method of identifying a subject having increased likelihood of responsiveness or sensitivity to an immunotherapy, said method comprising:
a) determining the level of texosomes associated with tumour cells that are unresponsive to cytokines;
wherein a level below a predetermined threshold is indicative of having an increased likelihood of responsiveness or sensitivity to the immunotherapy.

29) A method of identifying a subject having responsiveness or sensitivity to an immunotherapy, said method comprising:
a) determining the level of texosomes associated with tumour cells that are unresponsive to cytokines;
wherein a level below a predetermined threshold is indicative of having responsiveness or sensitivity to the immunotherapy.

30) The method according to any one of claims 27 to 29, wherein the immunotherapy is selected from the group consisting of: cytokine therapy, dendritic cell vaccines, adoptive T cell vaccines, peptide vaccines, checkpoint inhibitors and antibodies which induce the release of cytokines.

31) Immunotherapy for use in the treatment of cancer in a subject, wherein the subject has undergone texosome MHC-I profile analysis.

32) Cytokine therapy or HDAC inhibitors for use in the treatment of cancer in a subject, wherein the subject has undergone texosome MHC-I profile analysis.

33) A method of increasing the sensitivity rate of a cancer vaccines utilising HLA-A2 specific peptides to treat or prevent cancer in a patient population, said method comprising selecting a subpopulation, wherein the major histocompatibility complex profile of the total tumour burden has been determined using texosome analysis in the subpopulation and said subpopulation has:
a) a level below a predetermined threshold of texosomes associated with tumour cells having a loss of HLA-A2 expression.

34) A method of identifying a subject having increased likelihood of responsiveness or sensitivity to a cancer vaccine utilising a HLA-A2 specific peptide, said method comprising:
a) determining the level of texosomes associated with tumour cells having a loss of HLA-A2 expression;
Wherein a level below a predetermined threshold is indicative of having an increased likelihood of responsiveness or sensitivity to a cancer vaccine utilising a HLA-A2 specific peptide.

35) A method of identifying a subject having responsiveness or sensitivity to a cancer vaccine utilising a HLA-A2 specific peptide, said method comprising:
a) determining the level of texosomes associated with tumour cells having a loss of HLA-A2 expression;
Wherein a level below a predetermined threshold is indicative of having responsiveness or sensitivity to a cancer vaccine utilising a HLA-A2 specific peptide.

36) Cancer vaccines for use in the treatment or prevention of cancer in a subject, wherein the subject has undergone texosome MHC-I expression profile analysis.

37) A method of determining a treatment regime for a subject having cancer, said method comprising:
a) determining the major histocompatibility complex profile of the total tumour burden of said subject with cancer in accordance with the method of any one of claims 1 to 19;
b) administering a medicament based on the major histocompatibility complex profile of the total tumour burden of said subject.

38) A method of treating cancer in a subject identified as having an increased likelihood of responsiveness or sensitivity to an immunotherapy in accordance with the method of claim 28, comprising:
a) administering a therapeutically effective amount of a cytokine.

39) A method of treating or preventing cancer in a subject identified as having an increased likelihood of responsiveness or sensitivity to cancer vaccines utilising HLA-A2 specific peptides in accordance with the method of claim 34, comprising:
a) administering a therapeutically effective amount of a cancer vaccine utilising HLA-A2 specific peptide.

40) An isolated and enriched population of texosomes for which the MHC-I profile has been determined in accordance with the method of any one of claims 1 to 19 for use in diagnostics.

41) Use of an isolated and enriched population of texosomes for which the MHC-I profile has been determined in accordance with the method of any one of claims 1 to 19 for:
a) diagnosis;
b) prognosis;
c) determining treatment; or
d) preparing modulated texosomes.

42) A method of determining the HLA-G expression profile of a tumour of a subject with cancer comprising the steps of:
a) obtaining a physiological fluid sample comprising texosomes or a texosome-enriched sample of extracellular vesicles from a physiological fluid sample of the subject;
b) identifying the phenotypes of texosomes associated with tumour cells having HLA-G expression.
